(19) United States
(12) Patent Application Publication
(10) Pub. No.: US 2019/0130340 A1
(43) Pub. Date: May 2, 2019

(54) WORKER MANAGEMENT APPARATUS

(71) Applicant: Mitsubishi Electric Corporation, Chiyoda-ku (JP)

(72) Inventors: Ai TAKAMI, Tokyo (JP); Wataru KOSHI, Tokyo (JP); Hiroe TAKENAGA, Tokyo (JP); Hirokazu KAIEDA, Tokyo (JP)

(73) Assignee: Mitsubishi Electric Corporation, Chiyoda-ku (JP)

(21) Appl. No.: 16/092,150

(22) PCT Filed: Jan. 6, 2017
(86) PCT No.: PCT/JP2017/000290

§ 371 Date: Oct. 8, 2018

(30) Foreign Application Priority Data
Apr. 26, 2016 (JP) 2016-088285

(51) Int. Cl. G06Q 10/06; A61B 5/11

(52) U.S. Cl. CPC G06Q 10/063114; A61B 2503/20; A61B 5/1118

(57) ABSTRACT

A workload information management device includes a per-scene load calculation unit calculating the workload value that is an estimate of a workload on a worker in action scenes based on time-series vital sign information from start time to end time of an action of the worker in extracted action scenes. The workload information management device further includes a per-scene load accumulation unit accumulating worker information, scene information for identifying action scenes, and a workload value for the action scenes, which are associated with one another, and a multiple people load comparison unit determining whether a workload value for one of the action scenes is higher than a predetermined condition by comparing the workload value with those of multiple people accumulated in the per-scene load accumulation unit and associated with the same scene information as that on the one of the action scenes.

### FIELD

The present invention relates to a worker management apparatus for managing worker health and safety.

### BACKGROUND

Stress checks for workers became mandatory with the revision in December 2015 of the Industrial Health and safety Act, and thus employers; have recently been increasingly asked to understand the psychological and physical loads on employees. In particular, workers who work outdoors or who work in low-temperature environments in workplaces such as plants, factories, and construction sites are often engaged in work in an environment, with high psychological and physical loads compared with workers who perform sedentary work only. For this reason, health management of workers who work in workplaces such as plants, factories, and construction sites is considered to be more important. However, it is not easy for employers to objectively determine the workloads on workers and to manage worker's health.

Patent Literature 1 discloses a technique for recognizing the condition of a user on the basis of vital sign information and action information obtained from a group of sensors worn on the body of the user and then presenting the recognition results to the user. With the technique described in Patent Literature 1, it is possible to acquire, in relation to each other, an action history and vital sign information on the user by using a body-worn device and to then present advice on health management to

### CITATION LIST

### Patent Literature

Patent Literature 1: Japanese Patent Application Laid-open No. 2006-320735

### SUMMARY

### Technical Problem

However, with the technique described in Patent Literature 1, it is assumed that the health state of only one person is observed, and no consideration is given to a case where there are a plurality of people observed. For this reason, there is a problem that it is difficult to distinguish in the detection between a state where the majority of a plurality of workers are under a high load and an abnormal state where only a specific worker is under a high load even though the majority of the workers can perform the work without experiencing such a load.

The present invention has been made in view of the above, and it is an object of the present invention to obtain a worker management apparatus capable of detecting whether the workload on a worker is relatively high with respect to the workloads on a plurality of people who have performed the same work.

### Solution to Problem

In order to solve the above-described problems and to achieve the object, the worker management apparatus according to an aspect of the present invention includes a worker information collection device worn by a worker, the worker information collection device including a vital sign information measuring unit to collect time-series vital sign information on the worker during work; and an action information collection unit to collect work information including work, content of work performed by the worker and including a work start date and time and a work end date and time when the worker performs the work. The worker management apparatus further includes a workload information management device capable of communicating with the worker information collection device. The workload information management device includes a vital sign information accumulation unit to accumulate the time-series vital sign information; an action history accumulation unit to accumulate action information in which worker information indicating a worker who performs the work and the work information are associated with each other; an action segment, extraction unit to divide a series of actions of the work recorded in the work information into a plurality of action scenes on a basis of a change in content of the action in a series of actions of the work performed by the worker, and to extract the action scenes; and a per-scene load calculation unit, to calculate a workload value that is an estimate of a workload on the worker in the action scene on a basis of the time-series vital sign information from start time to end time of an action of the worker in the action scene extracted by the action segment extraction unit. The workload information management device further includes a per-scene load accumulation unit to accumulate the worker information, scene information for identifying the action scenes, and the workload value for the action scenes that are associated with one another; and a multiple people load comparison unit to compare the workload value for one of the action scenes with the workload value of a plurality of people that is accumulated in the per-scene load accumulation unit and associated with same scene information as the scene information on the one of the action scenes and to determine whether the workload value for the one of the action scenes is higher than a predetermined condition.

### Advantageous Effects of Invention

The present invention achieves an effect of obtaining a worker management apparatus capable of detecting whether the workload on a worker is relatively high with respect to the workloads on a plurality of people who have performed the same work.

### BRIEF DESCRIPTION OF DRAWINGS

FIG. 1 is a diagram illustrating a configuration of a worker management apparatus according to a first embodiment of the present invention.

FIG. 2 is a diagram illustrating an example of action information accumulated in an action history accumulation unit of a worker information collection device according to the first embodiment.

FIG. 3 is a diagram illustrating an example of vital sign information accumulated in a vital sign information accumulation unit of the worker information collection device according to the first embodiment.

FIG. 4 is a diagram illustrating an example of workload information accumulated in a per-scene load accumulation unit of the worker information collection device according to the first embodiment.

FIG. 5 is a block diagram illustrating an example of a hardware configuration of a computer for realizing each of a workload information management device and a workload information viewing device according to the first embodiment.

FIG. 6 is a block diagram illustrating an example of a hardware configuration of a wearable terminal for realizing the worker information collection device according to the first embodiment.

FIG. 7 is a flowchart illustrating an operation when the workload information management device according to the first embodiment receives action information from the worker information collection device.

FIG. 8 is a flowchart illustrating an operation when the workload information management device according to the first embodiment receives vital sign information from the worker information collection device.

FIG. 9 is a flowchart illustrating details of a procedure of Step S30, in which an action segment extraction unit extracts scenes from the action information, in the flowchart illustrated in FIG. 7.

FIG. 10 is a flowchart illustrating details of a procedure of Step S40, in which the per-scene load calculation unit calculates the workload value for each scene, in procedures illustrated in FIG. 7.

FIG. 11 is a flowchart, illustrating details of a procedure of Step S60, which is a determination step in a multiple people load comparison unit, in the procedures illustrated in FIG. 7.

FIG. 12 is a flowchart illustrating an operation when an action information collection unit of the worker information collection device according to the first embodiment transmits action information to the workload information management device.

FIG. 13 is a flowchart illustrating an operation when a vital sign information measuring unit of the worker information collection device according to the first embodiment transmits vital sign information to the workload information management device.

FIG. 14 is a flowchart illustrating an operation when a load information display control unit of the workload information viewing device according to the first embodiment acquires the determination result of a workload from the workload information management device, and warns a manager that there is a worker under a prominently high load.

FIG. 15 is a schematic view illustrating an example of display of a screen of the worker information collection device according to the first embodiment.

FIG. 16 is a schematic view illustrating an example of display of the screen of the worker information collection device according to the first embodiment.

FIG. 17 is a schematic view illustrating an example of display of the screen of the worker information collection device according to the first embodiment.

FIG. 18 is a schematic view illustrating an example of display of the screen of the worker information collection device according to the first embodiment.

FIG. 19 is a schematic view illustrating an example of display of the screen of the worker information collection device according to the first embodiment.

FIG. 20 is a diagram illustrating a configuration of a worker management apparatus according to a second embodiment.

FIG. 21 is a diagram illustrating an example of terminal information accumulated in a terminal information accumulation unit of a workload information management device according to the second embodiment.

FIG. 22 is a flowchart illustrating an operation of an instruction content distribution unit of the workload information management device according to the second embodiment.

FIG. 23 is a flowchart illustrating an operation of a load information display control unit off a workload information viewing device according to the second embodiment.

FIG. 24 is a flowchart illustrating an operation of an instruction content display control unit of a worker information collection device according to the second embodiment.

FIG. 25 is a schematic view illustrating an example of display of a screen of the workload information viewing device according to the second embodiment.

FIG. 26 is a schematic view illustrating an example of display of the screen of the workload information viewing device according to the second embodiment.

FIG. 27 is a schematic view illustrating an example of display of a screen of the worker information collection device according to the second embodiment.

FIG. 28 is a diagram illustrating a configuration of a worker management apparatus according to a third embodiment.

FIG. 29 is a diagram illustrating an example of worker position information accumulated in an action history accumulation unit of a workload information management device according to the third embodiment.

FIG. 30 is a diagram illustrating an example of external information accumulated in an external information accumulation unit of the workload information management device according to the third embodiment.

FIG. 31 is a flowchart illustrating an operation when the workload information management device according to the third embodiment receives worker position information from a worker information collection device.

FIG. 32 is a flowchart illustrating an operation when the workload information management device according to the third embodiment receives external information from an external information collection device.

FIG. 33 is a flowchart illustrating a procedure of extracting a scene performed by an action segment extraction unit of the workload information management device according to the third embodiment, on the basis of information accumulated in the action history accumulation unit and the external information accumulation unit.

FIG. 34 is a flowchart illustrating a procedure of extracting a scene performed by the action segment extraction unit of the workload information management device according to the third embodiment on the basis of action information and worker position information accumulated in the action history accumulation unit.

FIG. 35 is a flowchart illustrating an operation of collecting worker; position information and transmitting the worker position information to the workload information management device performed by the worker information collection device according to the third embodiment.

FIG. 36 is a flowchart illustrating an operation of collecting external information and transmitting the external information to the workload information management device performed by an external information measuring unit of the external information collection device according to the third embodiment.

### DESCRIPTION OF EMBODIMENTS

Hereinafter, a worker management apparatus according to embodiments of the present invention will be described in detail with reference to the drawings. The present invention is not limited to the embodiments.

### First Embodiment

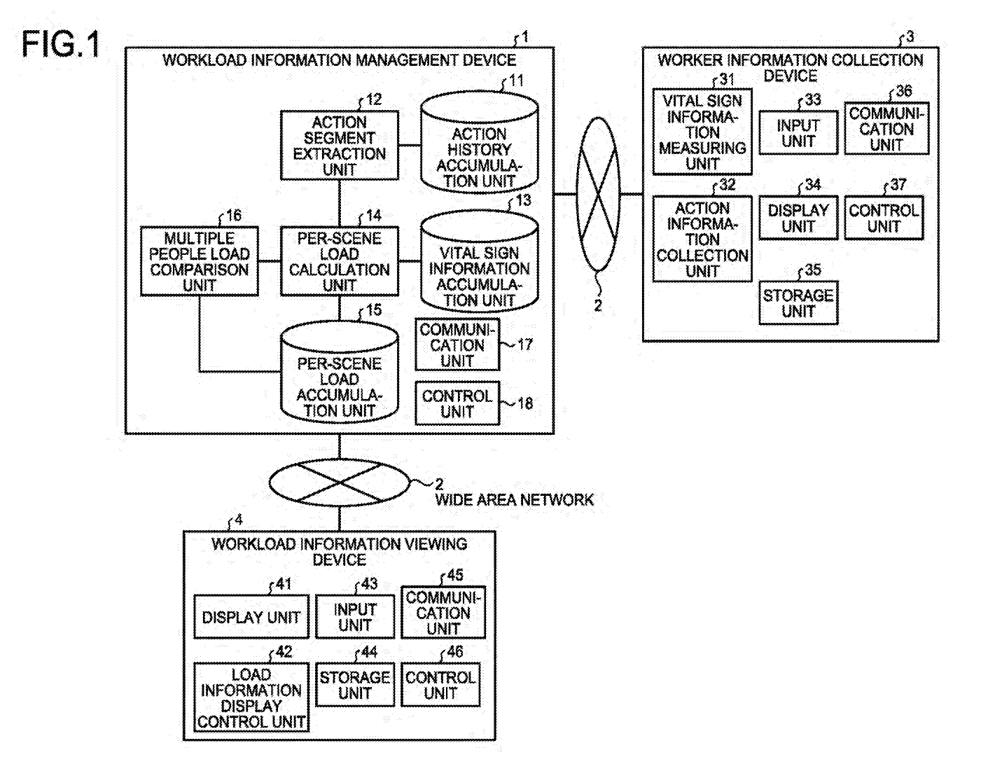

FIG. 1 is a diagram illustrating a configuration of a worker management apparatus according to a first embodiment of the present invention. The worker management apparatus according to the first embodiment includes a workload information management device 1; a worker information collection device 3 including a wristwatch-type wearable terminal; and a workload information viewing device 4. The workload information management device 1 accumulates action information and time-series vital sign information collected by the worker information collection device 3, and calculates the workload value in each scene in response to a request from the workload information viewing device 4.

First, the configuration of the workload information management device 1 will be described. The workload information management device 1 includes an action history accumulation unit 11; an action segment extraction unit 12; a vital sign information accumulation unit 13; a per-scene load calculation unit 14; a per-scene load accumulation unit 15; a multiple people load comparison unit 16; a communication unit 17; and a control unit 18.

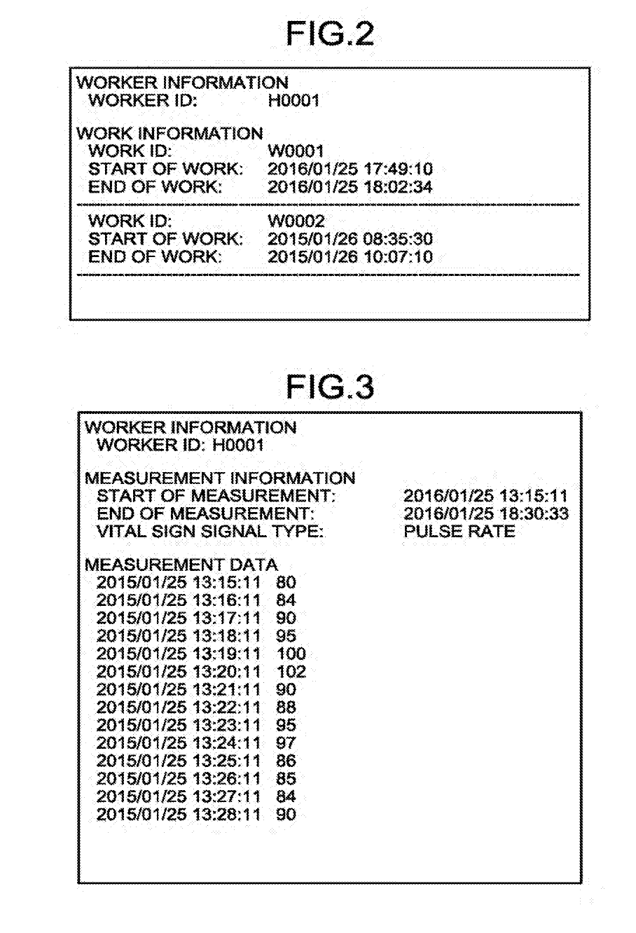

The action history accumulation unit 11 stores and accumulates the action information collected by the worker information collection device 3. FIG. 2 is a diagram illustrating an example of action information accumulated in the action history accumulation unit 11 of the worker information collection device 3 according to the first embodiment.

The action information is information on actions of workers and it includes worker information on a worker who wears the worker information collection device 3 and work information on work performed by the worker wearing the worker information collection device 3, which are associated with each other.

The worker information, is worker identification information with which a worker to be managed by the worker management apparatus can be uniquely identified and it includes worker identification (ID). In the first embodiment, the worker information is described by a combination of an English letter and numerals as “H0001”.

The work information includes a work ID with which work to be performed by a worker managed by the worker management apparatus can be uniquely identified, work start date and time, and work end date and time. In the first embodiment, the work ID is described by a combination of an English letter and numerals such as “W0001”.

The work start date and time is information with which date and time when the worker started work can be uniquely identified. In the first embodiment, year, month, day, hour, minute, and second are described therein, as “2016/1/25 17:49:10”. The work end date and time is information with which date and time when the worker ended the work can be uniquely identified. In the first embodiment, year, month, day, hour, minute, and second are described therein, as “2016/1/25 18:02:34”. When it is determined that only one work operation is performed on the same day, hour, minute, and second may be omitted from the work start date and time and the work end date and time.

On the basis of a change in the content of an action in a series of actions of the work performed by the worker, the action segment extraction unit 12 divides a series of actions of the work recorded in the work information into a plurality of action scenes and extracts the scenes. That is, the action segment extraction unit 12 divides the action of the worker obtained in the form of action information collected by the worker information collection device 3 into one or more action scenes on the basis of a predetermined action segment rule, and extracts the scenes.

The scene is obtained by dividing a series of actions of the worker obtained in the form of action information on the basis of the predetermined action segment rule. Here, the action segment refers to a point in the series of actions of the worker that can be regarded as a point before and after which the content of the action has changed. In the first embodiment, the action segment refers to the timing at which a phase of work has changed. For example, it is assumed that a series of actions by which a worker performs inspection includes a plurality of actions, i.e., movement from an office to an inspection site, actual inspection work, recording of inspection results, and movement from the inspection site to the office. In that case, the action segment indicates the timing of shifting from the movement to the inspection work, the timing of shifting to the recording of the inspection results after the inspection work, and the timing of starting the movement from the inspection site to the office after the recording of the inspection results. Thus, the action segment is a point where the content of the action in the series of actions of the work, performed by the worker has changed.

The vital sign information accumulation unit 13 stores and accumulates the time-series vital sign information collected by the worker information collection device 3. Hereinafter, the time-series vital sign information is sometimes simply referred to as vital sign information. FIG. 3 is a diagram illustrating an example of the vital sign information accumulated in the vital sign information accumulation unit 13 of the worker information collection device 3 according to the first embodiment.

The vital sign information is time-series vital sign data such as a heart rate, a pulse rate, an amount, of perspiration, and brain waves in a certain period of time obtained by various sensors mounted on the worker information collection device 3 and worn on the body of the worker. The vital sign information includes a measurement value, measurement time, and a vital sign signal type, which are associated with one another.

The measurement value is described as a numerical value such as a real number and an integer. The measurement time is time information for identifying measurement time of each measurement value. As for the measurement time, it is sufficient if the measurement time of each, measurement value can be identified. Thus, when a cycle at which a value is measured by a sensor worn on the body of the worker is fixed at one second, the measurement time can be identified for all the measurement values by only describing the measurement start, time, and thus, the description of the measurement time corresponding to each measurement value may be omitted.

The vital sign signal type is character string information indicating the type of a vital sign signal such as a heart rate, a pulse rate, an amount of perspiration, and brain waves. As long as the type of the vital sign signal can be numerically identified by a rule predetermined such that the heart rate is indicated by 0, the pulse rate is indicated by 1, and the amount of perspiration is indicated by 2, the vital sign signal type may be represented by a numerical value.

The per-scene load calculation unit 14 acquires, from the vital sign information accumulation unit 13, the vital sign information corresponding to the action scenes extracted by the action segment extraction unit 12, calculates the workload value for each worker and for each scene on the basis of the acquired vital sign information, and stores the value in the per-scene load accumulation unit 15.

The workload value indicates an estimate of physical and psychological loads experienced by one worker for one scene. In the first embodiment, the workload value is the ratio of the average value of measured values during work, which is expressed as a percentage, on the basis of the measurement value at rest of each worker of the vital sign signal type corresponding to the vital sign information collected by the worker information collection device 3. The measurement value at rest of each worker of the vital sign signal type corresponding to the vital sign information collected by the worker information collection device 3 is stored in advance in the per-scene load calculation unit 14 or another storage unit included in the workload information management device 1.

When the average value of the measurement values of the vital sign information on the worker during work is larger than the measurement value of the worker at rest, the workload value becomes larger than 100. On the other hand, when the average value of the measurement values of the vital sign information during work is smaller than the measurement value at rest, the workload value becomes smaller than 100. Any value may be used as the workload value as long as the magnitude of the workload values of two or more different workers or the workload values of the same worker at different times can be compared. For example, the average value of measurement values during work may be used as it is.

The per-scene load accumulation unit 15 accumulates, for each scene, workload information for each worker and for each scene calculated by the per-scene load calculation unit 14.

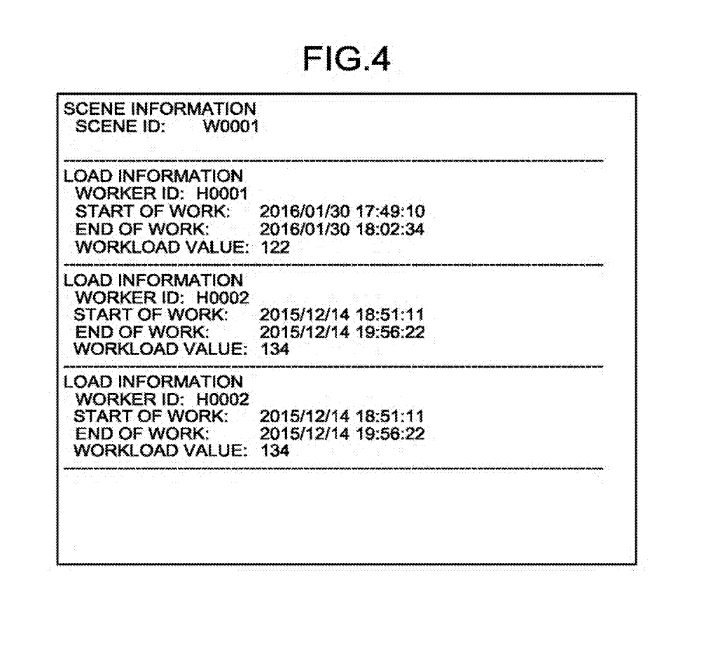

The workload information includes worker information, scene information, and a workload value, which are associated with one another. The scene information is identification information for identifying each scene, and it includes a scene ID with which the scene obtained by the action segment extraction unit 12 can be uniquely identified. In the first embodiment, the scene ID is equivalent to the work ID. FIG. 4 is a diagram illustrating an example of the workload information accumulated in the per-scene load accumulation unit 15 of the worker information collection device 3 according to the first embodiment.

The multiple people load comparison unit 16 compares the workload values that correspond to two or more workers in the same scene and are accumulated in the per-scene load accumulation unit IS, extracts a worker with a prominently high load value, and creates load information. The load information in the first embodiment is information including at least worker information on a worker having a prominently high workload value in a designated scene, i.e., a worker under a prominently high load. That is, the multiple people load comparison unit 16 compares a workload value for one action scene of a worker with workload values of a plurality of people, which are workload values associated with the same scene information as the scene information on the one action scene and accumulated in the per-scene load accumulation unit 15, to determine whether the workload value for the one action scene of the worker is higher than a predetermined condition.

The communication unit 17 wirelessly communicates with the worker information collection device 3 via a wide area network 2. The communication unit 17 receiver vital sign information and action information from a communication unit 36 of the worker information, collection device 3 described later. The communication unit 17 communicates with the workload information viewing device 4 via the wide area network 2. The control unit 18 provides overall control of the workload information management device 1 including control of each component in the workload information management device 1.

Next, the configuration of the worker information collection device 3 will be described. The worker information collection device 3 is worn on the body of a worker who performs work, collects action information and vital sign information on the worker, and transmits the collected action information and vital sign information to the workload information management device 1.

The worker information collection device 3 includes a vital sign information measuring unit 31, an action information collection unit 32, an input unit 33, a display unit 34, a storage unit 35, the communication unit 36, and a control unit 37. The components in the worker information collection device 3 can communicate with one another.

The vital, sign information measuring unit 31 includes various vital sign sensors for collecting the vital sign information on the worker and collects the vital sign information on the worker in a state where the worker information collection device 3 is worn on the body of the worker. Examples of the sensors included in the vital sign information measuring unit 31 include an electrocardiograph, a blood-pressure meter, a blood-saturation meter, a thermometer, and a perspiration meter. By using these sensors, the vital sign information measuring unit 31 can collect vital sign information such as the state of the heart, blood pressure, blood saturation level, body temperature, and perspiration state. The vital sign information measuring unit 31 transmits the vital sign information to the workload information management device 1 via the communication unit 36.

The action information collection unit 32 collects the action information on the worker on the basis of an input by the worker. The action information collection unit 32 transmits the action information to the workload information management device 1 via the communication unit 36.

The input unit 33 accepts inputs of various types of information from the worker and transmits the inputs to the control unit 37 or each component. The display unit 34 displays various types of information collected in the worker information collection device 3. The storage unit 35 stores various types of information in the worker information collection device 3. The communication unit 36 wirelessly communicates with the workload information management device 1 via the wide area network 2. The control unit 37 accepts various types of information and provides overall control of the worker information collection device 3 including control of each component in the worker information collection device 3. The control unit 37 controls the display of the display unit 34.

Next, the configuration of the workload information viewing device 4 will be described. The workload information viewing device 4 is operated by a manager who manages the load conditions of the workers. In response to an input by the manager, the workload information viewing device 4 requests the workload information management device 1 via the wide area network 2 to calculate the workload on a worker to be managed, and presents load information sent back from the workload information management device 1 via the wide area network 2 to the manager.

The workload information viewing device 4 includes a display unit 41; a load information display control unit 42; an input unit 43; a storage unit 44; a communication unit 45; and a control unit 46. The components in the workload information viewing device 4 can communicate with one another.

The display unit 41 displays the load information acquired from the workload information management device 1 and presents the load information to the manager. The display unit 41 displays a warning to the workers.

The load information display control unit 42 performs control such that the load information acquired from the workload information management device 1 is displayed on the display unit 41. The load information display control unit 42 performs control such that a warning is displayed for the workers.

The input unit 43 accepts inputs of various types of information from the manager. The storage unit 44 stores various types of information in the workload information viewing device 4. The communication unit 45 communicates with the workload information management device 1 via the wide area network 2. The control unit 46 provides overall control of the workload information viewing device 4 including control of each component in the workload information viewing device 4.

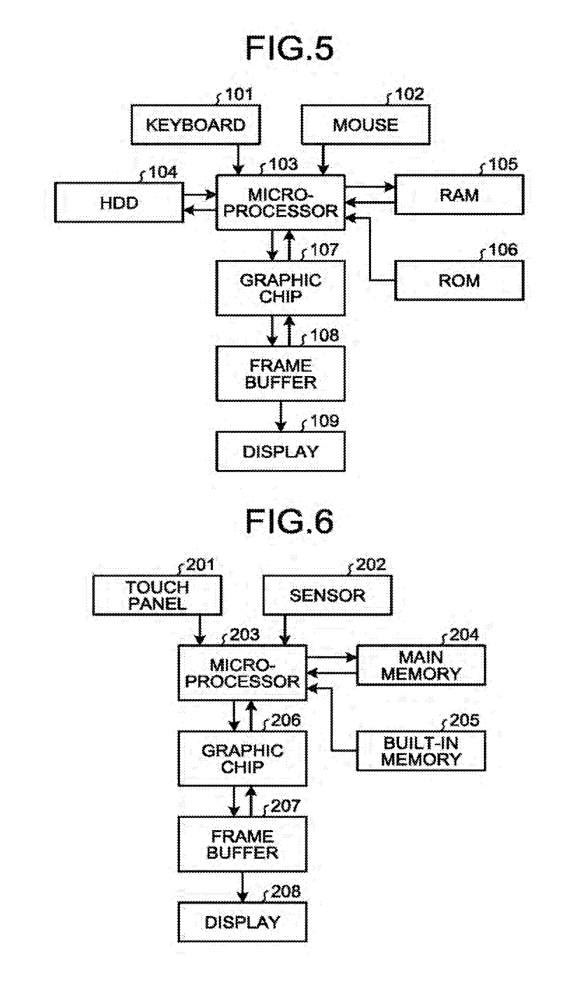

FIG. 5 is a block diagram illustrating an example of a hardware configuration of a computer for realizing each of the workload information management device 1 and the workload information viewing device 4 according to the first embodiment. The computer illustrated in FIG. 5 includes a keyboard 101, a mouse 102, a microprocessor 103, a hard disk drive (HDD) 104, a Random Access Memory (RAM) 105, a Read Only Memory (ROM) 106, a graphic chip 107, a frame buffer 108, and a display 109.

When each of the action history accumulation unit 11, the action segment extraction unit 12, the vital sign information accumulation unit 13, the per-scene load calculation unit 14, the per-scene load accumulation unit 15, the multiple people load comparison unit 16, the communication unit 17, and the control unit 16 of the workload information management device 1 is realized by the hardware configuration illustrated in FIG. 5, each component is realized by the microprocessor 103 executing a program stored in the ROM 106. A plurality of microprocessors 103 and a plurality of ROMs 106 may cooperate with each other to realize the above functions. Some of the functions of the above components may be implemented as an electronic circuit, and the other functions may be realized by using the microprocessor 103 and the ROM 106. By using the graphic chip 107 and the frame buffer 108, it is possible to perform a process more smoothly and at higher speed.

When each of the load information display control unit 42, the communication unit 45, and the control unit 46 of the workload information viewing device 4 is realized by the hardware configuration illustrated in FIG. 5, each component is realized by the microprocessor 103 executing a program stored in the ROM 106. A plurality of microprocessors 103 and a plurality of ROMs 106 may cooperate with each other to realize the above functions. Some of the functions of the above components may be implemented as an electronic circuit, and the other functions may be realized by using the microprocessor 103 and the ROM 106. The display unit 41 is realized as the display 109. The input unit 43 is realized as the keyboard 101, the mouse 102, or the display 109 having a touch panel function. The storage unit 44 is realized as the hard disk drive 104 or the RAM 105.

FIG. 6 is a block diagram illustrating an example of a hardware configuration of a wearable terminal for realising the worker information collection device 3 according to the first embodiment. In the present embodiment, the worker information collection device 3 is realized as a wristwatch-type wearable terminal. However, it is sufficient if the worker information collection device 3 is realized as a terminal that can be worn on the body, and the worker information collection device 3 may be realized as an underwear-type wearable terminal, for example. The illustrated terminal includes a touch panel 201, a sensor 202, a microprocessor 203, a main memory 204, a built-in memory 205, a graphic chip 206, a frame buffer 207, and a display 208.

When each of the action information collection unit 32, the communication unit 36, and the control unit 37 of the worker information collection device 3 is realized by the hardware configuration illustrated in FIG. 6, each component is realized by the microprocessor 203 executing a program stored in the built-in memory 205. A plurality of microprocessors 203 and a plurality of built-in memories 205 may cooperate with each other to realize the above functions. Some of the functions of the above components may be implemented as an electronic circuit, and the other functions may be realized by using the microprocessor 203 and the built-in memory 205. The vital sign information measuring unit 31 includes the sensor 202 and is realized by the microprocessor 203 executing a program stored in the built-in memory 205. The input unit 33 is realized as the touch panel 201. The display unit 34 is realized as the display 208. The storage unit 35 is realized as the built-in memory 205.

In the first embodiment, the worker information collection device 3 transmits the action information and the vital sign information to the workload information management device 1 via the wide area network 2, but it is not always necessary to perform the transmission via the wide area network 2. The action information and the vital sign information collected by the worker information collection device 3 may be accumulated in the built-in memory 205, and thereafter, the action information and the vital sign information may be duplicated or moved to the workload information management device 1 by using an external storage device such as a Universal Serial Bus (USB) memory.

In the first embodiment, the workload information management device 1 and the workload information viewing device 4 are connected to each other via the wide area network 2, but the workload information management device 1 and the workload information viewing device 4 may be realized on the same computer. When the workload information management device 1 and the workload information viewing device 4 are realized on the same computer, it is sufficient if a display unit is provided in the workload information management device 1 in order to realize a display function in the workload information viewing device 4.

In the first embodiment, the vital sign information measuring unit 31 and the action information collection unit 32 of the worker information collection device 3 are realized as the same wearable terminal, but this is not a limitation. All or a part of the vital sign information measuring unit 31 may be realized as another terminal such as a ring-type pulse sensor. When all or a part of the vital sign information measuring unit 31 is realized as another terminal, it is sufficient if the vital sign information measuring unit 31 uses wireless communication such as Bluetooth (registered trademark) or an external storage device such as a USB memory to acquire vital sign information.

Next, a flow of processes performed by each device in the worker management apparatus will be described.

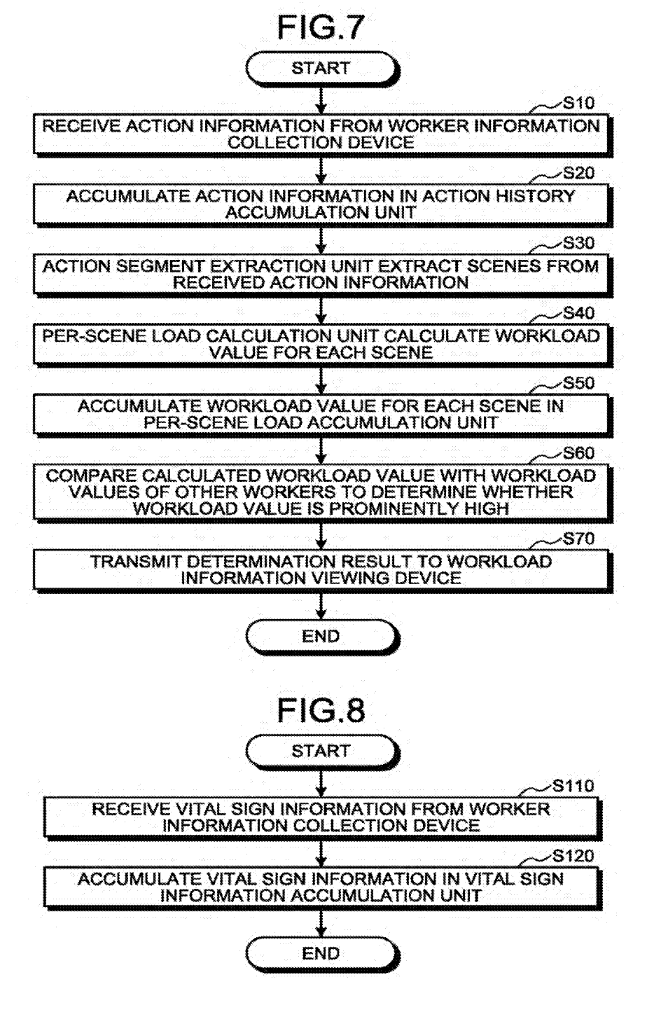

First, operations of the workload information management device 1 will be described with reference to the flowcharts of FIGS. 7 to 11.

FIG. 7 is a flowchart illustrating an operation when the workload information management device 1 according to the first embodiment receives action information from the worker information collection device 3.

In Step S10, the communication unit 17 of the workload information management device 1 receives action information from the communication unit 36 of the worker information collection device 3. The communication unit 17 transmits the received action information to the action history accumulation unit 11. In Step S20, the action history accumulation unit 11 stores and accumulates the received action information.

In Step S30, the action segment extraction unit 12 extracts action scenes from the action information newly stored in the action history accumulation unit 11. Next, in Step S40, the per-scene load calculation unit 14 calculates the workload value for each scene. Then, in Step S50, the per-scene load accumulation unit 15 stores and accumulates the workload value for each scene.

Next, in Step S60, the multiple people load comparison unit 16 compares the workload value calculated in Step S40 with the workload values of other workers who have performed the same scene to determine whether the workload value calculated in Step S40 is prominently high. That is, the workload value calculated in Step S40 is compared with the workload values of other workers who have performed the same scene, and thereby it is determined whether the workload value calculated in Step S40 is higher than a predetermined condition. For example, when the difference between the workload value calculated in Step S40 and the average value of the workload values of other workers who have performed the same scene is greater than or equal to a predetermined reference value, the workload value calculated in Step S40 is determined to be prominently high. The workload values of other workers who have performed the same scene are extracted by the multiple people load comparison unit 16 from the workload values of the workload information on other workers having the same scene information, i.e., the same scene ID, of the workload information accumulated in the per-scene load accumulation unit 15.

Then, in Step S70, the multiple people load comparison unit 16 transmits the determination result in Step S50 to the workload information viewing device 4 via the communication unit 17.

FIG. 8 is a flowchart illustrating an operation when the workload information management device 1 according to the first embodiment receives vital sign information from the worker information collection device 3.

In Step S110, the communication unit 17 of the workload information management device 1 receives vital sign information from the communication unit 36 of the worker information collection device 3. The communication unit 17 transmits the received vital sign information to the vital sign information accumulation unit 13. In Step S120, the vital sign information accumulation unit 13 stores and accumulates the received vital sign information.

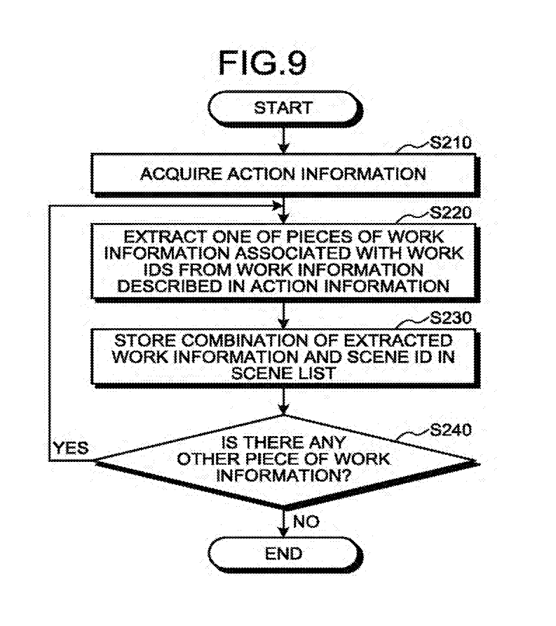

FIG. 9 is a flowchart illustrating details of the procedure of Step S30, in which the action segment extraction unit 12 extracts scenes from action information, in the flowchart illustrated in FIG. 7.

In Step S210, the action segment extraction unit 12 acquires the action information newly stored in the action history accumulation unit 11. Next, in Step S220, the action segment extraction unit 12 extracts work information associated with one of the work IDs of the works performed by the worker from the work information described in the acquired action information.

In the first embodiment, the scene ID is equivalent to the work ID. The action segment extraction unit 12 associates a scene ID with each work operation classified by a work ID with which work to be performed by a worker can be uniquely identified. That is, in the first embodiment, an action of the worker described in the action information is divided into scenes corresponding to the respective work operations classified by work IDs. The action segment extraction unit 12 extracts an action scene for each work operation classified by a work ID.

Next, in Step S230, the action segment extraction unit 12 stores a combination of a scene ID and the work information associated with one of the extracted work IDs in a new scene list. In the scene list, the worker information on the worker described in the action information is also stored in association with the combination of the scene ID and the work information associated with one of the work IDs. Next, in Step S240, the action segment extraction unit 12 determines whether there is another piece of work information that has not yet been divided as a scene in the acquired action information.

When there is another piece of work information, i.e., when Yes is determined in Step S240, the action segment extraction unit 12 returns to Step S220 and extracts the work information associated with one of the other work IDs. When there is no other piece of work information, i.e., when No is determined in Step S240, the action segment extraction unit 12 transmits the scene list to the per-scene load calculation unit 14, and ends the process of extracting a scene from the action information.

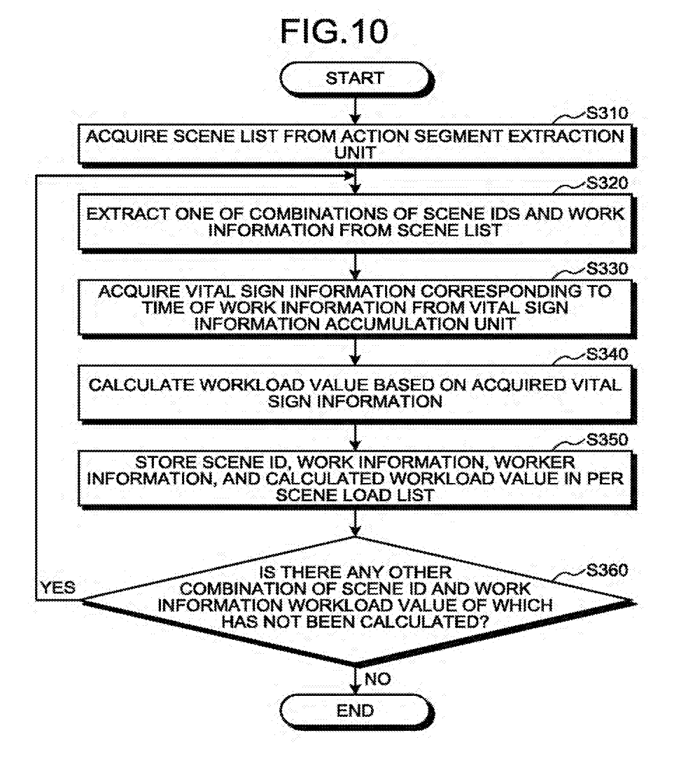

FIG. 10 is a flowchart illustrating details of the procedure of Step S40, in which the per-scene load calculation unit 14 calculates the workload value for each scene, in the procedure illustrated in FIG. 7.

In Step S310, the per-scene load calculation unit 14 acquires the scene list transmitted from the action segment extraction unit 12. Next, in Step S320, the per-scene load calculation unit 14 extracts one of the combinations of the scene IDs and the work information from the scene list.

Next, in Step S330, the per-scene load calculation unit 14 acquires vital sign information corresponding to the time indicated in the extracted work information from the vital sign information accumulation unit 13.

Next, in Step S340, the per-scene load calculation unit 14 calculates the workload value on the basis of the acquired vital sign information. Here, in the first embodiment, in the procedure of Step S340 of calculating the workload value on the basis of the acquired vital sign information, when the measurement value of each worker at rest of the vital sign signal type corresponding to the vital sign information collected by the worker information collection device 3 is denoted by Vᵣₑₛₜ, a workload value L can be expressed by the following equation (1).

$$\begin{matrix}[ Equation 1 ] & \\ L = \frac{A}{{V}_{rest}} \times 100 & ( 1 )\end{matrix}$$

However, when the measurement value at a certain time Tt between time Ts and time Te is denoted by Vt, an average value A of the measurement values can be expressed by the following equation (2). The time Ts and time Te are in units of seconds.

$$\begin{matrix}[ Equation 2 ] & \\ A = \frac{{\sum }_{t = Ts}^{Te} Vt}{{T}_{e} - {T}_{s}} & ( 2 )\end{matrix}$$

Next, in Step S350, the per-scene load calculation unit 14 stores the extracted scene ID, the work information, the worker information, and the calculated workload value in a new per-scene load list. Next, in Step S360, the per-scene load calculation unit 14 determines whether there is another combination of a scene ID and work information the workload value of which has not yet been calculated in the acquired scene list.

When there is another combination, i.e., when Yes is determined in Step S360, the per-scene load calculation unit 14 returns to Step S320 and extracts another combination of a scene ID and work information the workload value of which has not yet been calculated. When there is no other combination, i.e., when No is determined in Step S360, the per-scene load calculation unit 14 transmits the per-scene load list to the multiple people load comparison unit 16, and ends the process for calculating the workload value for each scene.

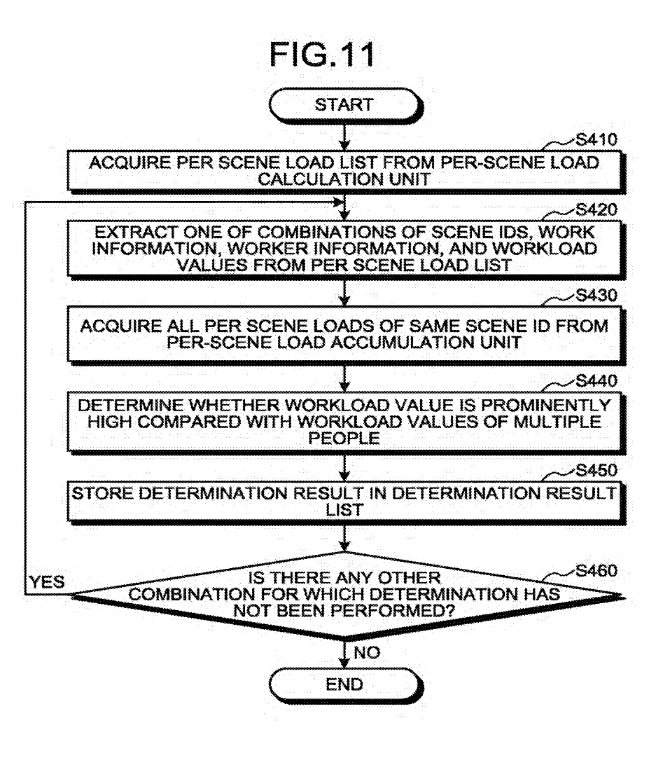

FIG. 11 is a flowchart illustrating details of the procedure of Step S60, which is a determination step in the multiple people load comparison unit 16, in the procedures illustrated in FIG. 7.

In Step S410, the multiple people load comparison unit 16 acquires the per-scene load list from the per-scene load calculation unit 14. Next, in Step S420, the multiple people load comparison unit 16 extracts one of the combinations of the scene IDs, the work information, the worker information, and the workload values from the per-scene load list.

Next, in Step S430, the multiple people load comparison unit 16 acquires, from the per-scene load accumulation unit 15, all the per scene loads of the same scene ID accumulated in the per-scene load accumulation unit 15, i.e., all the workload values corresponding to the same scene ID accumulated in the per-scene load accumulation unit 15.

Here, in the first embodiment, in the procedure of Step S430 of acquiring all the per scene loads of the same scene ID from the per-scene load accumulation unit 15, per scene loads of all the workers who have performed the work of the same scene ID are acquired.

Next, in Step S440, the multiple people load comparison unit 16 determines whether the workload value extracted in Step S420 is prominently high compared with the workload values of a plurality of people who have performed the work of the same scene ID. In Step S440, whether the workload value extracted in Step S420 is prominently high is determined on the basis of whether the workload value extracted in Step S420 corresponds to an outlier among the workload values or a plurality of people who have performed the work of the same scene ID.

Specifically, whether the extracted workload value corresponds to an outlier is determined using the algorithm as follows. N workload values are extracted, from the workload values of a plurality of people acquired in Step S430, in descending order of closeness to the workload value L extracted in Step S420. Among the extracted N workload values, a value furthest from L is expressed as a workload value Lf.

Whether the workload value L corresponds to an outlier compared with workload values of a plurality of people is determined on the basis of whether the difference between the workload value L and the workload value Lf is not less than a predetermined reference value D. When the difference between the workload value L and the workload value Lf is greater than or equal to the predetermined reference value D, it is determined that the workload value L corresponds to an outlier compared with the workload values of a plurality of people.

In the first embodiment, N is 5 and the reference value D is 20. However, values other than the above may be used since it is sufficient if the workload value L can be determined to be prominent when the number of values close to the workload value L in the workload values of a plurality of people in the past is small and can be determined not to be prominent when the number is not small.

The algorithm for determining whether the extracted workload value corresponds to an outlier may be a method other than the above method. For example, a workload value the difference of which from the workload value L is within a predetermined reference value Dd is extracted from the workload values of a plurality of people, and in a case where the number of workload values obtained by the extraction is smaller than or equal to a predetermined reference number Nd, the extracted workload value may be determined as an outlier.

Here, the predetermined reference value Dd is 25 and the predetermined reference number Nd is 10. However, values other than the above may be used since it is sufficient if the workload value L can be determined to be prominent when the number of values close to the workload value L in the workload values of a plurality of people in the past is small and can be determined not to be prominent when the number is not small.

Next, in Step S450, the multiple people load comparison unit 16 stores the determination result in Step S440 in a new determination result list. When determining that the workload value extracted in Step S420 is prominently high, the multiple people load comparison unit 16 stores the determination result that indicates that the workload value is prominently high in the determination result list. When not determining that the workload value extracted in Step S420 is prominently high, the multiple people load comparison unit 16 stores the determination result that indicates that the workload value is normal in the determination result list. Next, in Step S460, the multiple people load comparison unit 16 determines whether there is another combination of a scene ID, work information, worker information, and a workload value for which the determination in Step S440 has not been performed.

When there is another combination, i.e., when Yes is determined in Step S460, the multiple people load comparison unit 16 returns to Step S420 and extracts another combination of a scene ID, work information, worker information, and a workload value for which the determination in Step S440 has not yet been performed. When there is no other combination, i.e., when No is determined in Step S460, the multiple people load comparison unit 16 ends the process for determining whether the workload value stored in the per-scene load list is prominently high compared with the workload values of a plurality of people who have performed the work of the same scene ID.

In the first embodiment, in the procedure of Step S440, whether the worker corresponding to the worker information extracted in step S420, i.e., the worker whose workload value is determined, is included in a plurality of people whose workload values have been acquired in Step S430 is not considered. However, only the workload value of the worker whose workload value is determined may be extracted and applied to an algorithm for determining whether the workload value corresponds to the outlier described above. In that case, it is possible to exclude workload values of other people in the past, so that an effect is obtained where it is possible to detect a situation where the physical condition of the worker is prominently bad compared with the physical condition of the worker when performing similar work in the past.

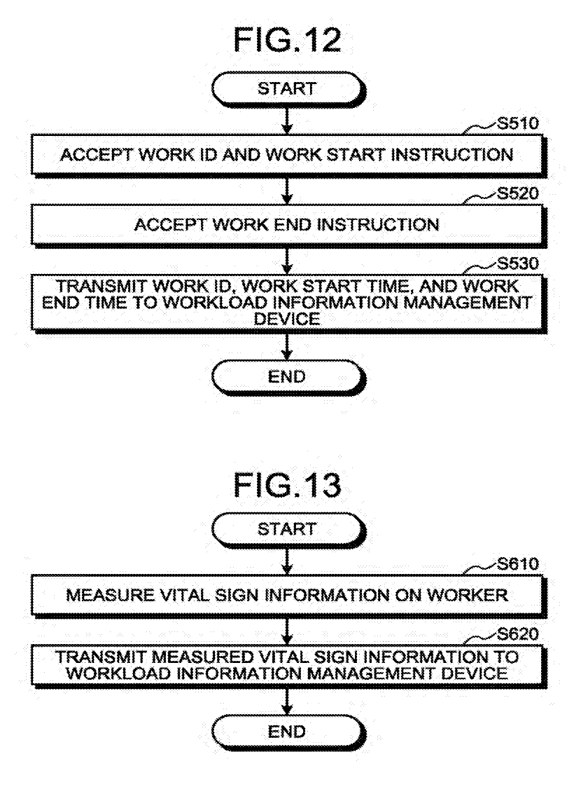

Next, operations of the worker information collection device 3 will be described with reference to flowcharts of FIGS. 12 and 13.

FIG. 12 is a flowchart illustrating an operation when the action information collection unit 32 of the worker information collection device 3 according to the first embodiment transmits action information to the workload information management device 1.

In Step S510, the action information collection unit 32 accepts a work ID and a work start instruction from the worker via the input unit 33. The work start instruction includes instruction information regarding what kind of work is to be performed. Upon accepting the work ID and the work start instruction, the action information collection unit 32 stores the work ID and the work start time in a memory in the action information collection unit 32. The action information collection unit 32 may store the work ID and the work start time in the storage unit 35.

Next, in Step S520, the action information collection unit 32 accepts a work end instruction from the worker via the input unit 33. Upon accepting the work end instruction, the action information collection unit 32 stores work end time in the memory in the action information collection unit 32. The action information collection unit 32 may store the work ID and the work end time in the storage unit 35.

Next, in Step S530, the action information collection unit 32 transmits the work ID, the work start time, and the work end time to the workload information management device 1 via the communication unit 36.

In the procedure illustrated in FIG. 12, the action information is transmitted to the workload information management device 1 at the timing when one work operation is ended, but the timing of transmitting the action information is not limited thereto. For example, multiple pieces of action information may be transmitted collectively at the timing when multiple work operations are ended. In that case, an effect is obtained where even when the worker information collection device 3 is in an environment where the worker information collection device 3 cannot communicate frequently with the workload information management device 1, action information can be collected and provided to the workload information management device 1.

FIG. 13 is a flowchart illustrating an operation when the vital sign information measuring unit 31 of the worker information collection device 3 according to the first embodiment transmits vital sign information to the workload information management device 1.

In Step S610, upon accepting a work start instruction via the input unit 33, the vital sign information measuring unit 31 activates a sensor and starts measuring the vital sign information on the worker. The vital sign information measuring unit 31 measures the vital sign information on the worker at a predetermined cycle.

Next, in Step S620, the vital sign information measuring unit 31 transmits the measured vital sign information on the worker to the workload information management device 1 via the communication unit 36. The vital sign information measuring unit 31 transmits the vital sign information to the workload information management device 1 every time the vital sign information measuring unit 31 measures the vital sign information on the worker at the predetermined cycle. When measuring a plurality of types of vital sign information using a plurality of sensors, the vital sign information measuring unit 31 transmits the plurality of types of vital sign information to the workload information management device 1.

Thereafter, upon accepting a work end instruction, the vital sign information measuring unit 31 deactivates the sensor and ends the measurement of the vital sign information on the worker.

In the procedure illustrated in FIG. 13, the vital sign in formation measuring unit 31 immediately transmits the measured vital sign information to the workload information management device 1, but the transmission timing of the vital sign information is not limited thereto. For example, the vital sign information measuring unit 31 stores and accumulates the vital sign information in the vital sign information measuring unit 31 or the storage unit 35 until the end of the day's work, and at the timing of the end of the day's work, the vital sign information for one day's work may be collectively transmitted to the workload information management device 1. In that case, an effect is obtained where even when the worker information collection device 3 is in an environment where the worker information collection device 3 cannot communicate frequently with the workload information management device 1, vital sign information can be collected and provided to the workload information management device 1.

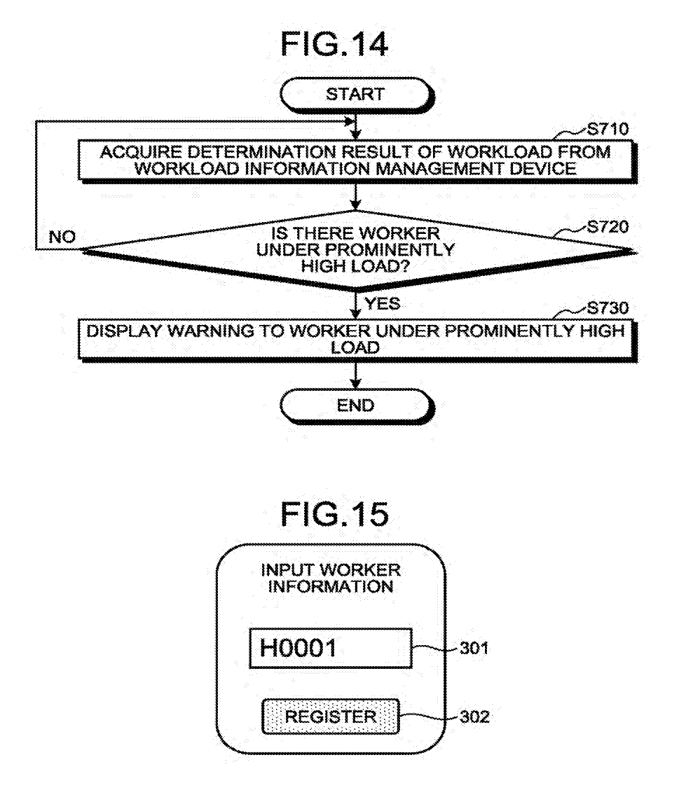

FIG. 14 is a flowchart illustrating an operation when the load information display control unit 42 of the workload information viewing device 4 according to the first embodiment acquires the determination result of the workload from the workload information management device 1, and warns the manager that there is a worker under a prominently high load.

In Step S710, the load information display control unit 42 acquires the determination result of the workload from the workload information management device 1 via the communication unit 45. Next, in Step S720, the load information display control unit 42 determines whether there is a worker under a prominently high load on the basis of the acquired determination result of the workload.

When there is a worker under a prominently high load, i.e., when Yes is determined in Step S720, the load information display control unit 42 performs control such that a warning is displayed on the display unit 41 for the worker under a prominently high load in Step S730. On the basis of the control of the load information display control unit 42, the display unit 41 displays the warning to the worker under a prominently high load.

When there is no worker under a prominently high load, i.e., when No is determined in Step S720, the load information display control unit 42 returns to Step S720.

Next, examples of operations of the worker management apparatus according to the first embodiment will be described. The worker management apparatus includes the workload information management device 1, the worker information collection device 3, and the workload information viewing device 4, which perform the above processes. First, an example of an operation will be described. In the operation, the worker information collection device 3 collects vital sign information and transmits the vital sign information to the workload information management device 1, and then the workload information management device 1 accumulates the vital sign information.

First, immediately before the worker performs work, the following procedures are executed.

(Procedure 1-1) Activation of Worker Information Collection Device 3

When the worker who wears the worker information collection device 3 on the body turns on the power of the worker information collection device 3, the control unit 37 of the worker information collection device 3 accepts an activation start request, and the control unit 37 activates the worker information collection device 3.

(Procedure 1-2) Start of Collection of Vital Sign Information

When the worker information collection device 3 is activated, the vital sign information measuring unit 31 activates a sensor and starts measuring vital sign information on the worker.

(Procedure 1-3) Transmission of Vital Sign Information

The vital sign information measuring unit 31 transmits the measured vital sign information on the worker to the workload information management device 1 via the communication unit 36.

Next, the workload information management device 1 executes the following procedures.

(Procedure 2-1) Reception of Vital Sign Information

The communication unit 17 of the workload information management device 1 receives the vital sign information transmitted from the communication unit 36 of the worker information collection device 3. The communication unit 17 transmits the received vital sign information to the vital sign information accumulation unit 13.

(Procedure 2-2) Accumulation of Vital Sign Information

The vital sign information accumulation unit 13 stores and accumulates the received vital sign information.

Next, an example of an operation will be indicated. In the operation, the worker information collection device 3 collects action information and transmits the action information to the workload information management device 1, then the workload information management device 1 calculates the workload value, and when the workload is prominently high compared with workload values of a plurality of people, a warning, is displayed on the workload information viewing device 4. The processes of the above-described (procedure 1-1) to (procedure 1-3), (procedure 2-1), and (procedure 2-2) are performed in parallel.

The following procedures are executed immediately before the worker performs work.

(Procedure 3-1) Activation of Worker Information Collection Device 3

(Procedure 3-1) is the same procedure as the above (procedure 1-1), and the control unit 37 activates the worker information collection device 3. Upon activating the worker information collection device 3, the control unit 37 shifts to a mode for accepting an input of the worker ID by the worker, and displays a screen for accepting an input of the worker ID on the display unit 34.

The worker information collection device 3 can accept, an input of worker information and an input of work information by the worker. The inputs of these pieces of information are realized by a GUI component for input accepting an input operation of a user, the GUI component for input being displayed on the touch panel 201 included in the worker information collection device 3 or the touch panel 201 connected to the worker information collection device 3, as the input unit 33.

(Procedure 3-2) Input of Worker Information

When the worker inputs the worker ID as worker information from the touch panel 201, the GUI component for input, accepts the worker ID and transmits the worker ID to the control unit 37. FIG. 15 illustrates an example of display of a screen of the display unit 34 at this point in time. FIG. 15 is a schematic view illustrating an example of display of the screen of the worker information collection device 3 according to the first embodiment. In the example of a screen illustrated in FIG. 15, the worker inputs the worker ID in a worker ID input area 301 and touches a register button 302, and thereby the input of the worker ID is accepted and transmitted to the control unit 37. Upon accepting the worker ID, the control unit 37 displays a work information input screen on the display unit 34.

(Procedure 3-3) Input of Work Information

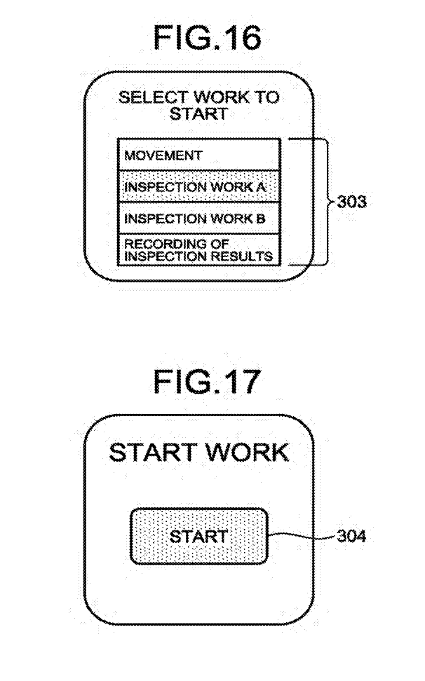

When the worker inputs information on the work to be performed from the touch panel 201, the GUI component for input accepts the information on the work. FIG. 16 illustrates an example of display of the screen of the display unit 34 at this point in time. FIG. 16 is a schematic view illustrating an example of display of the screen of the worker information collection device 3 according to the first embodiment. In the example of a screen illustrated in FIG. 16, multiple work selection buttons 303 for making a selection from multiple work types are displayed on the display 208, and the worker selects any one of the work selection buttons 303.

(Procedure 3-4) Wait for Start of Work

When the worker touches any one of the work selection buttons 303, for example, the work selection button 303 for selecting inspection work A, the control unit 37 switches the screen of the display unit 34 to display a screen for accepting start of work on the display unit 34. FIG. 17 illustrates an example of display of the screen of the display unit 34 at this point in time. FIG. 17 is a schematic view illustrating an example of display of the screen of the worker information collection device 3 according to the first embodiment. In the example of a screen illustrated in FIG. 17, a start button 304 for selecting start of work is displayed on the display 208.

(Procedure 3-5) Start of Work

When the worker touches the start button 304 arranged on the screen illustrated in FIG. 17, the work start information and the work ID are transmitted to the action information collection unit 32. Upon accepting the work start information and the work ID, the action information collection unit 32 stores work start time.

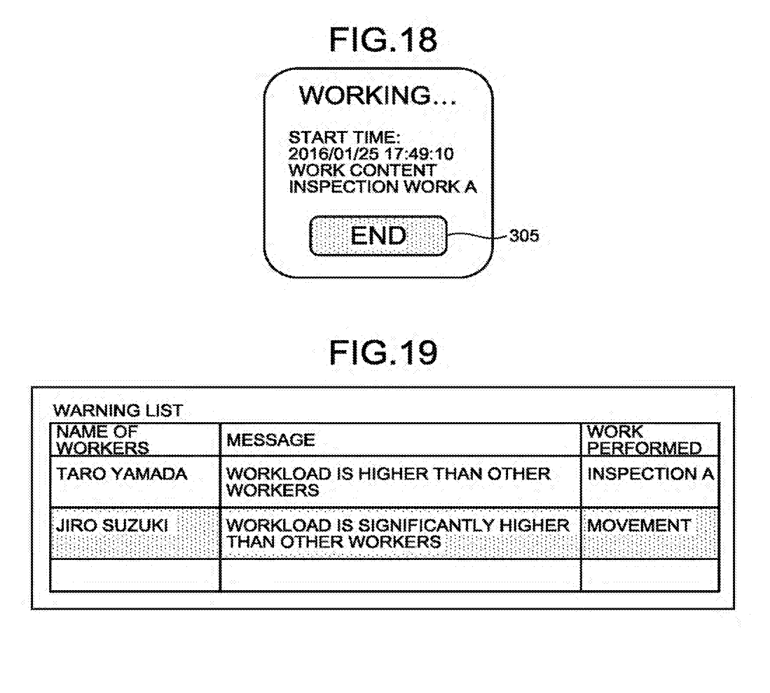

When the worker touches the start button 304 arranged on the screen illustrated in FIG. 17, the work start information is transmitted to the control unit 37. Upon accepting the work start information, the control unit 37 shifts to a mode for accepting the end of the work, and switches the screen of the display unit 34 to display a screen for accepting the end of the work on the display unit 34. FIG. 18 illustrates an example of display of the screen of the display unit 34 at this point in time. FIG. 18 is a schematic view illustrating an example of display of the screen of the worker information collection device 3 according to the first embodiment. In the example of a screen illustrated in FIG. 18, an end button 305 for selecting start of work is displayed on the display 208.

Thereafter, the following procedures are executed immediately after the worker completes the work.

(Procedure 4-1) Notification of End of Work

When the worker touches the end button 305 arranged on the screen illustrated in FIG. 18, the work end information is transmitted to the action information collection unit 32 and indicated to the action information collection unit 32. Upon accepting the work end information, the action information collection unit 32 stores work end time.

(Procedure 4-2) Transmission of Action Information

Thereafter, the action information collection unit 32 transmits the work ID, the work start time, and the work end time, which are pieces of action information, to the workload information management device 1.

Next, the following procedures are executed by the workload information management device 1.

(Procedure 5-1) Reception of Action Information

The workload information management device 1 receives the work ID, the work start time, and the work end time, which are pieces of action information. The received action information is stored and accumulated in the action history accumulation unit 11.

(Procedure 5-2) Determination of Workload

In the workload information management device 1, the action segment extraction unit 12 extracts action scenes from the received action information. The per-scene load calculation unit 14 calculates the workload value for each extracted scene. The multiple people load comparison unit 16 determines whether the calculated workload is prominently high compared with the workloads on a plurality of people.

(Procedure 5-3) Transmission of Determination Result

The workload information management device 1 transmits to the workload information viewing device 4, the determination result of the workload, i.e., the determination result as to whether the calculated workload value is prominently high compared with the workloads on a plurality of people.

Next, the following procedures are executed by the workload information viewing device 4.

(Procedure 6-1) Reception of Determination Result

The workload information viewing device 4 receives the determination result of the workload from the workload information management device 1.

(Procedure 6-2) Display of Warning

The workload information viewing device 4 displays a warning on the display unit 41 when there is a worker under a prominently high load on the basis of the determination result. FIG. 19 illustrates an example of display of the screen of the display unit 34 at this point in time. FIG. 19 is a schematic view illustrating an example of display of the screen of the worker information collection device 3 according to the first embodiment. In the example of a screen illustrated in FIG. 19, names of workers, messages to the workers, and work performed are displayed.

In the worker management apparatus according to the first embodiment described above, the worker information collection device 3 collects action information and vital sign information on workers during work, and calculates workload values of the workers on the basis of the collected vital sign information. Then, by comparing the calculated workload values of the workers with workload values of multiple workers who have performed the same work in the past, the worker management, apparatus according to the first embodiment can detect a worker under a prominently high workload and notify the manager of that effect. As a result, the manager can objectively know of an abnormal state where, although the majority of other workers who have performed the same work do not experience a high load, a worker experiences a prominently high load when doing the work due to reasons such as poor physical condition. As a result, the manager can know which worker requires attention in terms of health control.

The worker management apparatus according to the first embodiment can divide the action information on the worker into scenes on the basis of the predetermined action segment rule, and calculate the workload value of the worker for each scene on the basis of the vital sign information. Thus, the worker management apparatus according to the first embodiment can extract work conforming to the predetermined rule as the same scene from a series of work operations performed by each of a plurality of workers. As a result, the worker management apparatus according to the first embodiment can detect, for each scene, through comparison with workload values of a plurality of people associated with the same scene, an abnormal state where, although it is work that the majority of other workers do not experience a high load, a worker experiences a prominently high load due to reasons such as poor physical condition, and it can then notify the manager of that effect.

Furthermore, the action segment extraction unit 12 according to the first embodiment can classify similar work operations in the actions of the workers as the same scene when the work operations conform to a predetermined rule, regardless of whether the work operations have the same work content. As a result, the worker management apparatus according to the first embodiment can compare the workload values of workers who have performed similar work, in addition to workers who have performed the same work.

Therefore, according to the worker management apparatus in the first embodiment, a workload value can be calculated on the basis of the vital sign information on the worker mesasured during work, and the calculated workload value can be compared with the workload values of a plurality of people accumulated in the past when the same work or similar work is performed, and accordingly, it is possible to detect an abnormal state where among multiple workers, the majority of the workers do not experience a load but only a specific worker experiences a high load, and to objectively determine the workloads on the workers.

The worker management apparatus according to the first embodiment is suitable for management of health and safety of maintenance staff of large scale facilities represented by plants, workers at factories and construction sites.

The determination result obtained by the workload information management device 1 is transmitted to the workload information viewing device 4 promptly. Alternatively, for example, the determination result obtained by the workload information management device 1 can be provided to the worker at the end of the day and be used for health management and for raising the awareness of health management for the worker. In that case, the worker can view the provided determination result obtained by the workload information management device 1 on a device that can display the determination result, such as a personal computer or a terminal owned by the worker.

### Second Embodiment

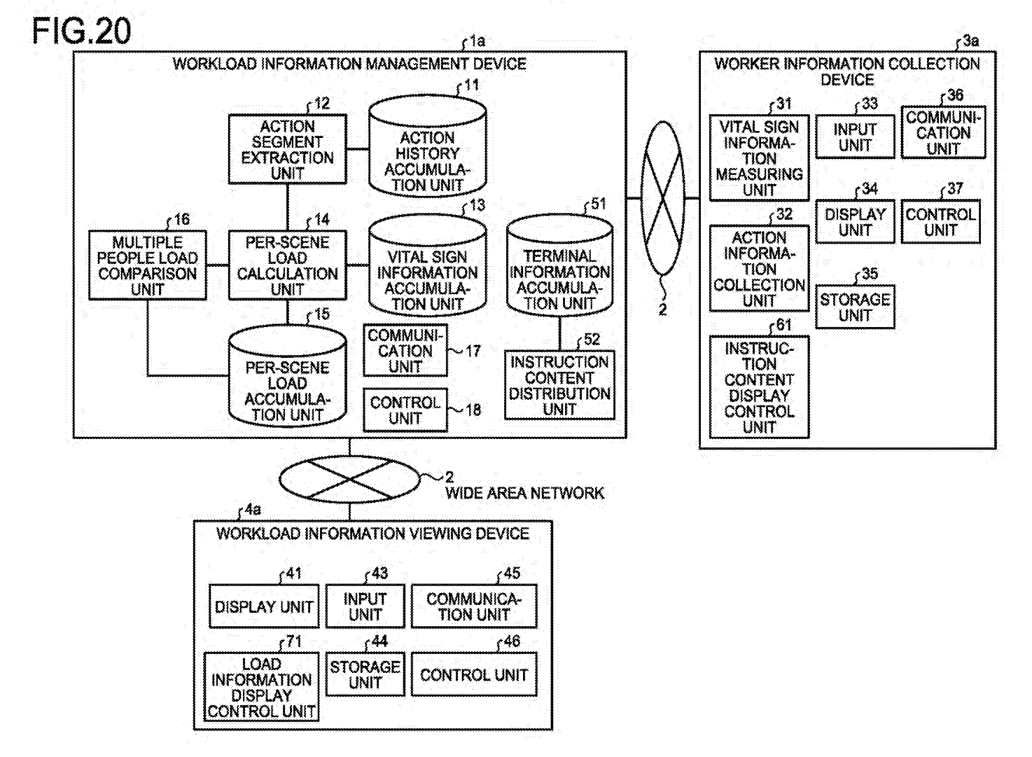

In a second embodiment, a case where a manager transmits an instruction to reduce a workload will be described. FIG. 20 is a diagram illustrating a configuration of a worker management apparatus according to the second embodiment. The worker management apparatus according to the second embodiment includes a workload information management device 1a; a worker information collection device 3a; and a workload information viewing device 4a. The wide area network 2 is similar to that in the first embodiment.

The workload information management device 1a has the same configuration and function as the workload information management device 1 according to the first embodiment except that the workload information management device 1a further includes a terminal information accumulation unit 51 and an instruction content distribution unit 52.

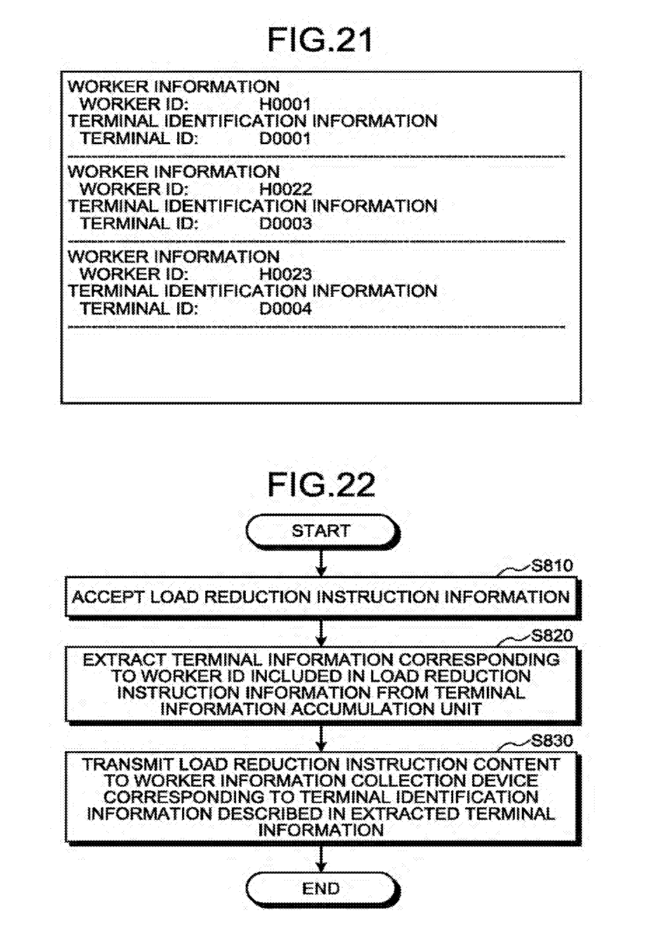

The terminal information accumulation unit 51 stores and accumulates terminal information. The terminal information includes worker information on a worker who wears the worker information collection device 3a and terminal identification information on a terminal that realizes the worker information collection device 3a, which are associated with each other. The terminal identification information is identification information with which the terminal of the worker information collection device 3a managed by the worker management apparatus according to the second embodiment can be uniquely identified, and it includes a terminal ID. In the second embodiment, the terminal identification information is described by a combination of an English letter and numerals as “D0001”. FIG. 21 is a diagram illustrating an example of the terminal information accumulated in the terminal information accumulation unit 51 of the workload information management device 1a according to the second embodiment.

The instruction content distribution unit 52 distributes instruction content in instruction information transmitted from the workload information viewing device 4a to the worker information collection device 3a. Specifically, the instruction content distribution unit 52 accepts load reduction instruction information transmitted from the workload information viewing device 4a via the communication unit 17. Here, the load reduction instruction information is instruction information instructing the worker to reduce a load, and is information including a worker ID and load reduction instruction content. The load reduction instruction content is content of an instruction to reduce a load to the worker, and is a character string transmitted from the workload information viewing device 1a in the second embodiment. The instruction content distribution unit 52 distributes the load reduction instruction content included in the load reduction instruction information to the worker information collection device 3a worn by the worker designated in the load reduction instruction information via the communication unit 17. On the basis of the worker ID included in the load reduction instruction information, and the terminal information corresponding to the worker ID accumulated in the terminal information accumulation unit 51, the instruction content distribution unit 52 distributes the load reduction instruction content to the worker information collection device 3a worn by the worker.

The worker information collection device 3a has the same configuration and function as the worker information collection device 3 according to the first embodiment except that the worker information collection device 3a further includes an instruction content display control unit 61. The instruction content display control unit 61 receives, via the communication unit 36, the load reduction instruction information transmitted from the workload information viewing device 4a via the workload information management device 1a, and performs control such that the load reduction instruction content included in the load reduction instruction information is displayed on the display unit 34.

The workload information viewing device 4a has the same configuration and function as the workload information viewing device 4 according to the first embodiment except that the workload information viewing device 4a includes a load information display control unit 71 instead of the load information display control unit 42. In addition to the function of the load information display control unit 42, the load information display control unit 71 has the following functions. The load information display control unit 71 accepts, from the manager via the input unit 43, an instruction to transmit the load reduction instruction information instructing a worker under a high load for whom a warning has been displayed on the display unit 41 to reduce the load. The load information display control unit 71 transmits the accepted load reduction instruction information to the worker information collection device 3a via the workload information management device 1a.

Next, a flow of processes executed by each device in the worker management apparatus according to the second embodiment will be described. A characteristic operation of each device in the worker management apparatus according to the second embodiment will be described below.

First, an operation of the instruction content distribution unit 52 of the workload information management device 1a according to the second embodiment will be described with reference to the flowchart of FIG. 22. FIG. 22 is a flowchart illustrating the operation of the instruction content distribution unit 52 of the workload information management device 1a according to the second embodiment.

In Step S810, the instruction content distribution unit 52 accepts load reduction instruction information. Next, in Step S820, the instruction content distribution unit 52 extracts terminal information corresponding to a worker ID included in the load reduction instruction information from the terminal information accumulation unit 51. Next, in Step S830, the instruction content distribution unit 52 transmits load reduction instruction content included in the load reduction instruction information to the worker information collection device 3a corresponding to terminal identification information described in the extracted terminal information.

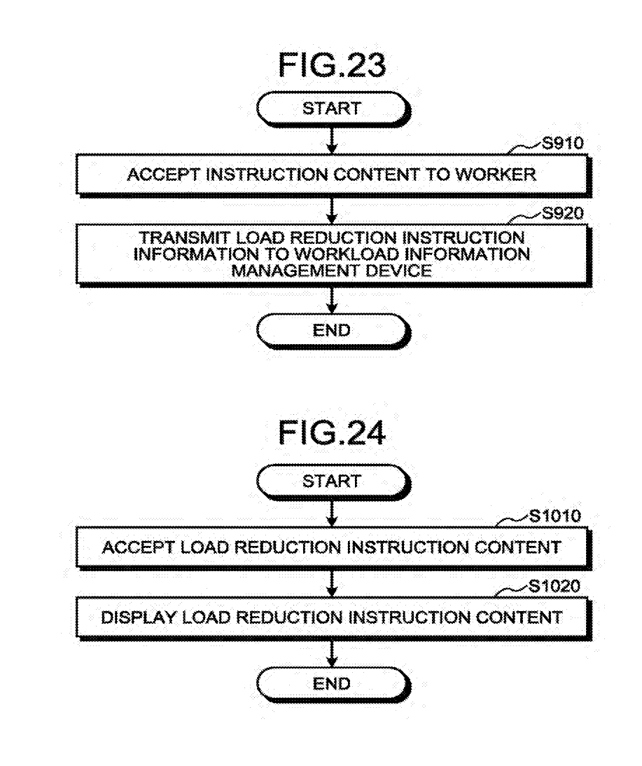

Next, an operation of the load information display control unit 71 of the workload information viewing device 4a will be described with reference to the flowchart of FIG. 23. FIG. 23 is a flowchart illustrating the operation of the load information display control unit 71 of the workload information viewing device 4a according to the second embodiment.

In the workload information viewing device 4a, the load information display control unit 71 receives the determination result of a workload from the workload information management device 1a as described with reference to the flowchart of FIG. 14 in the first embodiment. The display unit 41 displays a warning when there is a worker under a prominently high load on the basis of the determination result.

Then, in Step S910, the load information display control unit 71 accepts, from the manager via the input unit 43, instruction content of a load reduction instruction to the worker for whom the warning has been displayed on the display unit 41. Next, in Step S920, the load information display control unit 71 transmits the load reduction instruction information including the accepted instruction content of the load reduction instruction and the worker ID of the worker to the workload information management device 1a via the communication unit 45.

Next, an operation of the instruction content display control unit 61 of the worker information collection device 3a will be described with reference to the flowchart of FIG. 24. FIG. 24 is a flowchart illustrating the operation of the instruction content display control unit 61 of the worker information collection device 3a according to the second embodiment.

In Step S1010, the instruction content display control unit 61 accepts the load reduction instruction content transmitted from the workload information management device 1a. Next, in Step S1020, under the control of the instruction content display control unit 61, the display unit 34 displays the load reduction instruction content.

Next, an example of an operation of the worker management apparatus according to the second embodiment will be described. The worker management apparatus includes the workload information management device 1a, the worker information collection device 3a, and the workload information viewing device 4a, which perform the above processes. Here, a description will be given of an example of a series of operations in which the workload information viewing device 4a according to the second embodiment accepts a load reduction instruction from the manager and the worker information collection device 3a displays load reduction instruction content. The operation described below is performed after (procedure 6-2), which has been described in the operation example in the first embodiment and in which a warning is displayed on the display unit 41 when there is a worker under a prominently high load.

(Procedure 7-1) Transmission of Load Reduction Instruction Information

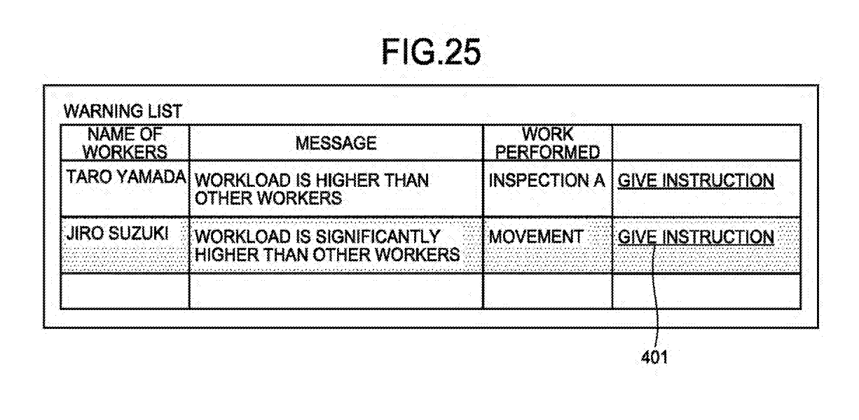

The load information display control unit 71 displays a warning to a worker under a prominently high load on the display unit 41, and then accepts an input of the load reduction instruction content. FIG. 25 illustrates an example of display of a screen of the display unit 41 at this point in time. FIG. 25 is a schematic view illustrating an example of display of the screen of the workload information viewing device 4a according to the second embodiment. In the example of a screen illustrated in FIG. 25, an instruction selection button 401 for selecting a process to give a load reduction instruction is added to the example of a screen illustrated in FIG. 19 in the first embodiment. When the instruction selection button 401 is selected, the load information display control unit 71 displays a screen for accepting the selection of the load reduction instruction on the display unit 41.

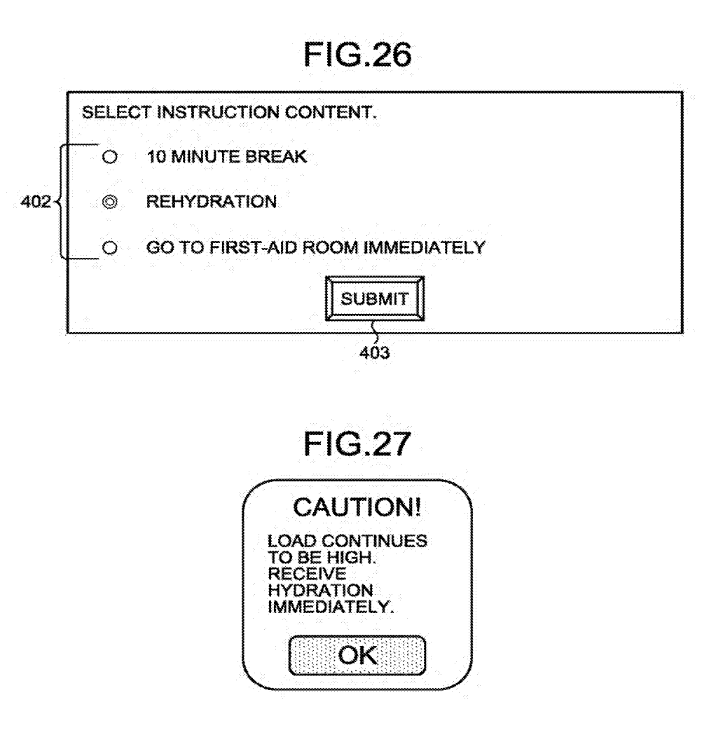

FIG. 26 illustrates an example of display of the screen of the display unit 41 at this point in time. FIG. 26 is a schematic view illustrating an example of display of the screen of the workload information viewing device 4a according to the second embodiment. In the example of a screen illustrated in FIG. 26, among multiple instruction content selection buttons 402 for selecting content of the load reduction instruction, the instruction content selection button 402 for desired instruction content is selected and a submit button 403 is touched. As a result, the load information display control unit 71 accepts the content of the load reduction instruction and a transmission instruction, and transmits load reduction instruction information including the worker ID of the selected worker and the load reduction instruction content to the workload information management device 1a via the communication unit 45. As the load reduction instruction content, a predetermined character string corresponding to the accepted instruction content of the instruction content selection button 402 is employed.

Thereafter, in the workload information management device 1a, the following procedures are executed.

(Procedure 8-1) Reception of Load Reduction Instruction Information

The workload information management device 1a receives the load reduction instruction information from the workload information viewing device 4a.

(Procedure 8-2) Extraction of Terminal Information

The instruction content distribution unit 52 extracts terminal information on a terminal corresponding to the load reduction instruction information from the terminal information accumulation unit 51.

(Procedure 8-3)

The instruction content distribution unit 52 transmits the load reduction instruction content included in the load reduction instruction information to the worker information collection device 3a realized by the terminal corresponding to the terminal information extracted in (procedure 8-2).

Thereafter, in the worker information collection device 3a, the following procedures are executed.

(Procedure 9-1) Reception of Load Reduction Instruction Information

The instruction content display control unit 61 receives the load reduction instruction content transmitted from the workload information management device 1a.

(Procedure 9-2) Display of Instruction Content

Under the control of the instruction content display control unit 61, the display unit 34 displays the load reduction instruction content. FIG. 27 illustrates an example of display of the screen of the display unit 34 at this point in time. FIG. 27 is a schematic view illustrating an example of display of the screen of the worker information collection device 3a according to the second embodiment.

In addition to the effects of the worker management apparatus according to the first embodiment, the worker management apparatus according to the second embodiment described above enables the manager to give an instruction to reduce a workload to a worker under a high workload, and therefore, it is possible to individually give a special instruction to reduce a workload only to the worker under a prominently high load compared with other workers. Then, the content of the instruction given by the manager can be presented to the worker by the worker information collection device 3a.

Therefore, according to the worker management apparatus in the second embodiment, it is possible for the manager of the workers to specify a worker determined to be under a prominently high load and to transmit an instruction to reduce a workload to the worker.

### Third Embodiment

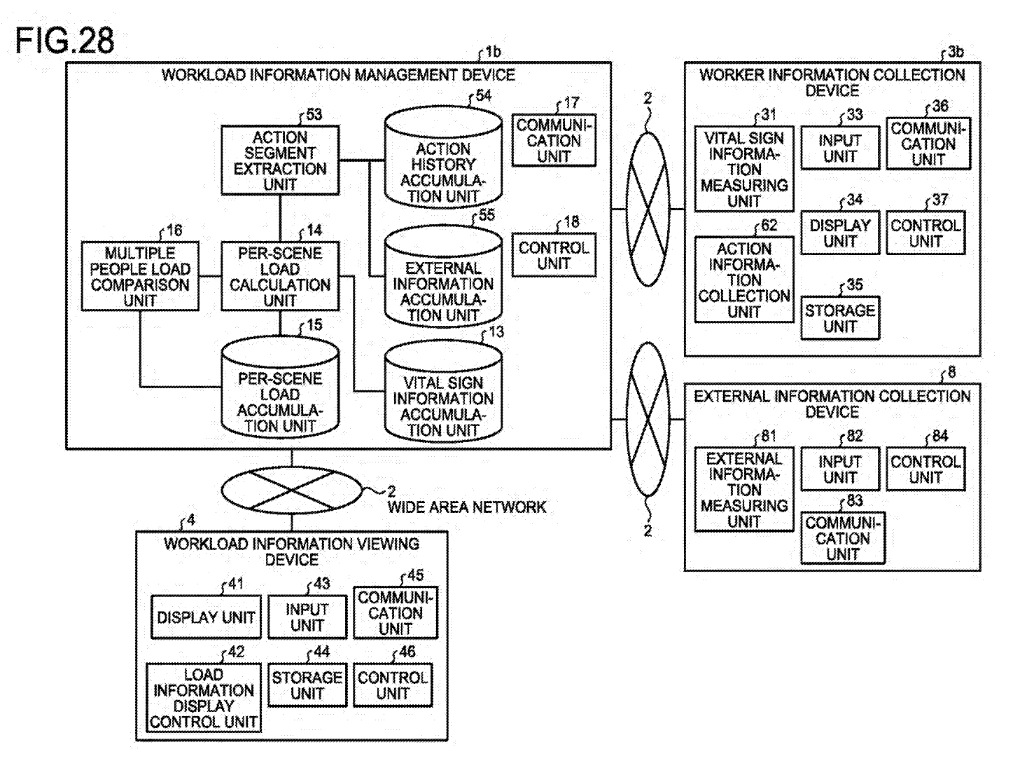

FIG. 28 is a diagram illustrating a configuration of a worker management apparatus according to a third embodiment. The worker management apparatus according to the third embodiment includes a workload information management, device 1b; a worker information collection device 3b; the workload information viewing device 4; and an external information collection device 8. The wide area network 2 is similar to that in the first embodiment.

The workload information management device 1b has the same configuration and function as the workload information management device 1 according to the first embodiment except that the workload information management device 1b includes an action segment extraction unit 53 instead of the action segment extraction unit 12, includes an action history accumulation unit 54 instead of the action history accumulation unit 11, and further includes an external information accumulation unit 55.

On the basis of action information and worker position information collected by the worker information collection device 3b and external information collected by the external information collection device 8, the action segment extraction unit 53 divides an action of a worker obtained in the form of action information collected by the worker information collection device 3b into one or more scenes on the basis of a predetermined action segment rule, and extracts the scenes.

The action history accumulation unit 54 receives, via the communication unit 17, the action information and the worker position information collected by the worker information collection device 3b, and then stores and accumulates these pieces of information. The worker position information is information for identifying the position of the worker during work, and it includes worker information on the worker wearing the worker information collection device 3b, collection information, and position information indicating the position of the worker during work, which are associated with one another. The worker information is similar to that in the first embodiment.

The collection information is information with which date and time when collection of the position information was started and date and time when the collection of the position information was ended can be uniquely identified, and it includes the date and time when collection of the position information was started and the date and time when the collection of the position information was ended.

The position information includes measurement time and measured position data, which are associated with each other. As for the measurement time, it is sufficient if the measurement time of each measurement value can be identified. Thus, when a cycle at which a value is measured by a sensor that measures the position of the worker during work is fixed at one second, for example, the measurement time can be identified for all the measurement values by only describing the measurement start time and thus, the description of the measurement time corresponding to each measurement value may be omitted.

The position data is information for uniquely identifying the position of the worker. In the third embodiment, the position data includes a combination of latitude and longitude. When the work site can be divided into several areas to which names are assigned, the names of the areas may be used for the position data.

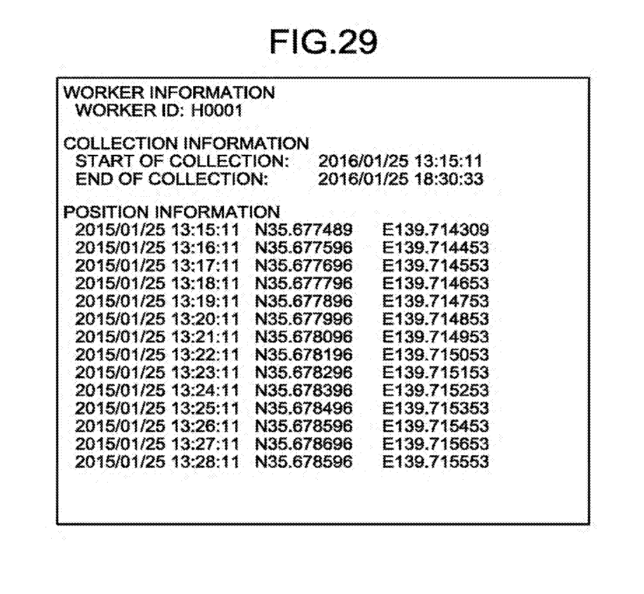

FIG. 29 is a diagram illustrating an example of the worker position information accumulated in the action history accumulation unit 54 of the workload information management device 1b according to the third embodiment. Among the position information illustrated in FIG. 29, N35.677489 indicates 35.677489 degrees north latitude and E139.714309 indicates 139.714309 degrees east longitude.

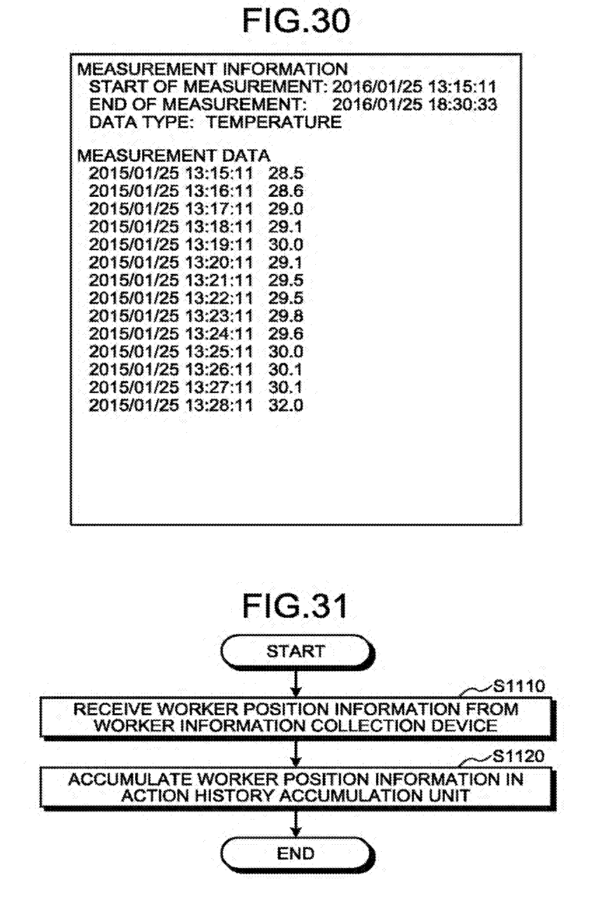

The external information accumulation unit 55 receives, via the communication unit 17, external information collected by the external information collection device 8, and then stores and accumulates the external information. Here, the external information is information indicating an external environmental condition where the worker performs work, and is described in a form in which measurement information indicating a condition at the time of measurement and measurement data are associated with each other. The measurement information includes time when the measurement was started, time when the measurement was ended, and a data type indicating the type of the measured data. The data type is character string information indicating the type of measured data such as temperature, humidity, altitude, and precipitation. FIG. 30 is a diagram illustrating an example of external information accumulated in the external information accumulation unit 55 of the workload information management device 1b according to the third embodiment.

The worker information collection device 3b has the same configuration and function as the worker information collection device 3 according to the first embodiment except that the worker information collection device 3b includes an action information collection unit 62 instead of the action information collection unit 32. The action information collection unit 62 includes a position sensor and collects worker position information by the position sensor in addition to the action information collected by the action information collection unit 32 in the first embodiment. The action information collection unit 62 transmits the collected worker position information to the workload information management device 1b via the communication unit 36.

The external information collection device 8 includes an external information measuring unit 81, an input unit 82, a communication unit 83, and a control unit 84. The external information measuring unit 81 measures external information by using a sensor arranged at a work site. That is, the external information measuring unit 81 collects measurement, data measured by the sensor arranged at the work site via the communication unit 83. The external information measuring unit 81 transmits the collected external information to the workload information management device 1b via the communication unit 83. The external information is information indicating an environmental condition including at least one of temperature, humidity, altitude, and precipitation.

The input unit 82 accepts inputs of various types of information from the worker and transmits the inputs to the control unit 84 or each component. The communication unit 83 wirelessly communicates with the sensor arranged at the work site and the workload information management device 1b via the wide area network 2. The control unit 84 provides overall control of the external information collection device 8 including control of each component in the external information collection device 8.

The external information collection device 8 can be realized by a computer having the hardware configuration illustrated in FIG. 5. When each of the external information measuring unit 81, the communication unit 83, and the control unit 84 of the external information collection device 8 is realized by the hardware configuration illustrated in FIG. 5, each component is realized by the microprocessor 103 executing a program stored in the ROM 106. A plurality of microprocessors 103 and a plurality of ROMs 106 may cooperate with each other to realize the above functions. Some of the functions of the above components may be implemented as an electronic circuit, and the other functions may be realized by using the microprocessor 103 and the ROM 106.

Next, a flow of processes executed by each device in the worker management apparatus according to the third embodiment will be described. A characteristic operation of each device in the worker management apparatus according to the third embodiment will be described below.

First, an operation of the action history accumulation unit 54 of the workload information management device 1b according to the third embodiment will be described with reference to the flowchart of FIG. 31. FIG. 31 is a flowchart illustrating the operation when the workload information management device 1b according to the third embodiment receives the worker position information from the worker information collection device 3b.

In Step S1110, the communication unit 17 of the workload information management device 1b receives the worker position information from the communication unit 36 of the worker information collection device 3b. The communication unit 17 transmits the received worker position information to the action history accumulation unit 54. In Step S1120, the action history accumulation unit 54 stores and accumulates the received worker position information.

Next, an operation of the external information accumulation unit 55 of the workload information management device 1b according to the third embodiment will be described with reference to the flowchart of FIG. 32. FIG. 32 is a flowchart illustrating the operation when the workload information management device 1b according to the third embodiment receives external information from the external information collection device 8.

In Seep S1210, the communication unit 17 of the workload information management device 1b receives external information from the communication unit 83 of the external information collection device 8. The communication unit 17 transmits the received external information to the external information accumulation unit 55. In Step S1220, the external information accumulation unit 55 stores and accumulates the received external information.

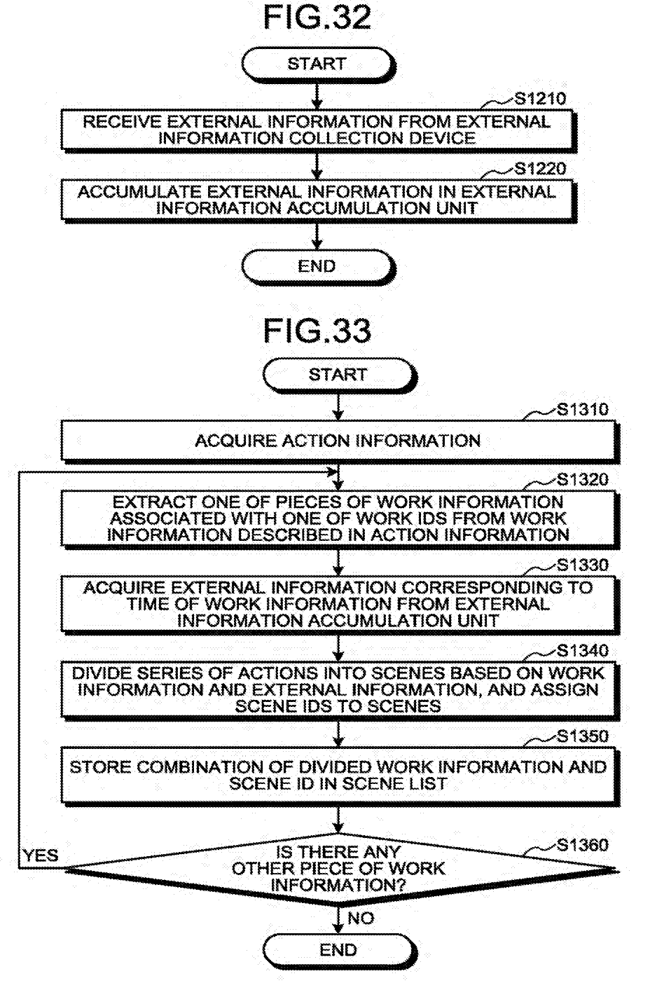

Next, an operation of the action segment extraction unit 53 of the workload information management device 1b according to the third embodiment will be described with reference to the flowchart of FIG. 33. FIG. 33 is a flowchart illustrating a procedure of extracting a scene performed by the action segment extraction unit 53 of the workload information management device 1b according to the third embodiment on the basis of information accumulated in the action history accumulation unit 54 and the external information accumulation unit 55.

First, prior to Step S1310, Steps S10 and S20 described with reference to the flowchart of FIG. 7 in the first embodiment are executed.

After Step S20, in Step S1310, the action segment extraction unit 53 acquires the action information newly stored in the action history accumulation unit 54. Next, in Step S1320, the action segment extraction unit 53 extracts work information associated with one of the work IDs of the works performed by the worker from the work information described in the acquired action information.

Next, in Step S1330, the action segment extraction unit 53 acquires external information corresponding to the time indicated in the extracted work information from the external information accumulation unit 55. Next, in Step S1340, the action segment extraction unit 53 divides a series of actions of the worker obtained in the form of action information into scenes on the basis of the extracted work information and the acquired external information, more specifically, divides a series of actions of the worker in a work time zone included in the extracted work information into scenes, and assigns a different scene ID to each of the divided scenes. Here, the external information is used as a predetermined action segment.

As an example, when external information is used as an action segment, it is possible to divide the extracted work information into scenes, i.e., to divide a series of actions of the worker in the work time zone included in the extracted work information into scenes, for example, depending on which temperature range of multiple preset temperature ranges the temperature as external information belongs to. When the temperature belongs to two different temperature ranges in the time zone from the work start time to the work end time described in the extracted work information, the extracted work information is divided into two scenes. That is, the series of actions of the worker in the work time zone included in the extracted work information is divided into two scenes. Then, different scene IDs are assigned to the scenes. When the temperature belongs to one temperature range in the time zone from the work start time to the work end time described in the extracted work information, the extracted work information is not divided into scenes and one scene ID is assigned thereto. That is, the series of actions of the worker in the work time zone, included in the extracted work information is not divided into scenes, and one scene ID is assigned thereto.

When the extracted work information is divided into multiple scenes, the work time zone included in the extracted work information is also divided for each scene. That is, each of the divided scenes is associated with the information on the work start time and the work end time in :he corresponding divided scene. Then, for each of the divided scenes, a combination of work information, a scene ID, and worker information is stored in a new scene list. As the work information, the work ID in the extracted work information and the information on the work start time and work end time in each scene are stored in the new scene list.

The action segment extraction unit 53 stores in advance temperature thresholds for dividing work information into multiple scenes. Examples of the temperature thresholds include 25 degrees, 30 degrees, and 35 degrees. On the basis of the extracted work information, the information on the temperature as the acquired external information, and the temperature thresholds, the action segment extraction unit 53 can divide a series of actions of the worker into multiple scenes at the time when the temperature information exceeds each temperature threshold value. Thus, as the multiple preset temperature ranges in this example, there are four temperature ranges: 25 degrees or lower; higher than 25 degrees and up to 30 degrees; higher than 30 degrees and up to 35 degrees; and higher than 35 degrees. The above-described temperature threshold values are merely an example and it is sufficient if the temperature threshold values are appropriately set. The action segment extraction unit 53 stores in advance thresholds for other types of external information in a similar manner, and thereby the action segment extraction unit 53 can divide the series of actions of the worker into multiple scenes on the basis of the thresholds.

Hereinafter, an example in which external information is used as an action segment will be more specifically described. In the external information accumulation unit 55, the temperature of the work site is stored as external information. Then, the work information is divided into scenes on the basis of the average of temperatures from the work start time to the work end time described in the extracted work information. In the third embodiment, in the cases where the temperature is lower than 0 degrees, where the temperature is higher than or equal to 0 degrees and lower than 10 degrees, where the temperature is higher than or equal to 11 degrees and lower than 20 degrees, where the temperature is higher than or equal to 20 degrees and lower than 30 degrees, and where the temperature is higher than or equal to 30 degrees, the scene IDs are set to 0, 1, 2, 3, and 4, respectively. It is sufficient it the scene IDs are assigned so as to take different values depending on the difference in external environmental conditions of the work site, and the threshold values for assigning the scene IDs may be set more finely or roughly than those in the above method.

In the third embodiment, as described above, the scene IDs are assigned on the basis of the data of temperatures described in the external information, but another type of external information such as humidity, altitude, or precipitation may also be used. Even in the case where another type of external information is used, it is sufficient if the same scene ID is assigned to similar external environmental conditions in a similar manner to the above case. In the work information on the divided scenes, the work ID and the work start time and work end time in each scene are associated with each other.

Next, in Step S1350, the action segment extraction unit 53 stores a combination of work information and a scene ID in the new scene list for each of the divided scenes. The action segment extraction unit 53 stores the work ID in the extracted work information and the information on the work start time and the work end time in each scene in the new scene list as work information. In the scene list, the worker information on the worker described in the action information is also stored in association with the combination, of each piece of work information divided into scenes and the scene ID. Next, in Step S1360, the action segment extraction unit 53 determines whether there is another piece of work information associated with the work ID of the work performed by the worker in the acquired action information.

When there is another piece of work information, i.e., when Yes is determined in Step S1360, the action segment extraction unit 53 returns to Step S1320 and extracts another piece of work information associated with one of the work IDs of the works performed by the worker from the work information described in the acquired action information. When there is no other piece of work information, i.e., when No is determined in Step S1360, the action segment extraction unit 53 transmits the scene list to the per-scene load calculation unit 14, and ends the process of extracting a scene from the action information.

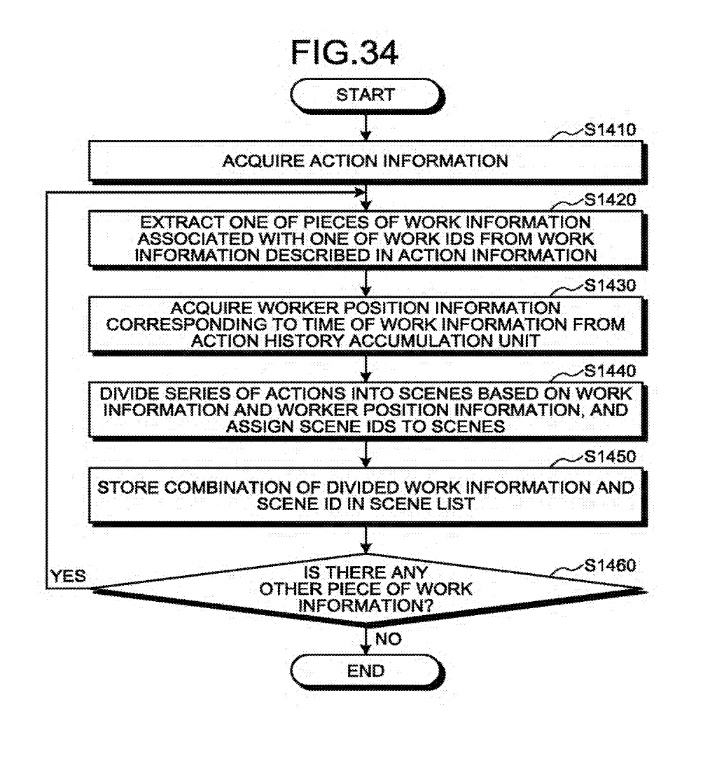

Next, another operation of the action segment extraction unit 53 of the workload information management device 1b according to the third embodiment will be described with reference to the flowchart of FIG. 34. FIG. 34 is a flowchart illustrating a procedure of extracting a scene performed by the action segment extraction unit 53 of the workload information management device 1b according to the third embodiment, on the basis of action information and worker position information accumulated in the action history accumulation unit 54.

First, prior to Step S1410, Steps S10 and S20 described with reference to the flowchart of FIG. 7 in the first embodiment are executed.

After Step S20, in Step S1410, the action segment extraction unit 53 acquires the action information newly stored in the action history accumulation unit 64. Next, in Step S1420, the action, segment extraction unit 53 extracts work information associated with one of the work IDs of the works performed by the worker from the work information described in the acquired action information.

Next, in Step S1430, the action segment extraction unit 53 acquires worker position information corresponding to the time indicated in the extracted work information from the action history accumulation unit 54. Next, in Step S1440, the action segment extraction unit 53 divides a series of actions of the worker obtained in the form of action information into scenes on the basis of the extracted work information and the acquired worker position information, more specifically, divides a series of actions of the worker in a work time zone included in the extracted work information into scenes, and assigns a different scene ID to each of the divided scenes. Here, the worker position information is used as a predetermined action segment.

As an example, when worker position information is used as an action segment, it is possible to divide the extracted work information into scenes, i.e., to divide a series of actions of the worker in the work time zone included in the extracted work information into scenes, for example, depending on which area of multiple preset areas the position of the worker identified by the worker position information belongs to. When the position of the worker identified by the worker position information belongs to two different areas in the time zone from the work start time to the work end time described in the extracted work information, the extracted work information is divided into two scenes. That is, the series of actions of the worker in the work time zone included in the extracted work information is divided into two scenes. Then, different scene IDs are assigned to the scenes. When the position of the worker identified by the worker position information belongs to one area in the time zone from the work start time to the work end time described in the extracted work information, the extracted work information is not divided into scenes, and one scene ID is assigned thereto. That is, the series of actions of the worker in the work time zone included in the extracted work information is not divided into scenes, and one scene ID is assigned thereto.

In the third embodiment, as described above, the scene ID is assigned on the basis of which area the position of the worker described in the worker position information belongs to, but the work information may be divided into scenes by other rules related to the position of the worker. Even when another rule of scene division based on the worker position information is used, it is sufficient if the same scene ID is assigned to the conditions where the positions of the workers are close to each other in a similar manner to the above case. In the work information on the divided scenes, the work ID and the work start time and work end time in each scene are associated with each other.

Next, in Step S1450, the action segment extraction unit 53 stores a combination of each piece of work information divided into scenes and a scene ID in the new scene list. In the scene list, the worker information on the worker described in the action information is also stored in association with the combination of each piece of work information divided into scenes and the scene ID. Next, in Step S1460, the action segment extraction unit 53 determines whether there is another piece of work information associated with the work ID of the work performed by the worker in the acquired action information.

When there is another piece of work information, i.e., when Yes is determined in Step S1460, the action segment extraction unit 53 returns to Step S1420 arid extracts another piece of work information associated with one of the work IDs of the works performed by the worker from the work information described in the acquired action information. When there is no other piece of work information, i.e., when No is determined in Step S1460, the action segment, extraction unit 53 transmits the scene list to the per-scene load calculation unit 14, and ends the process of extracting a scene from the action information.

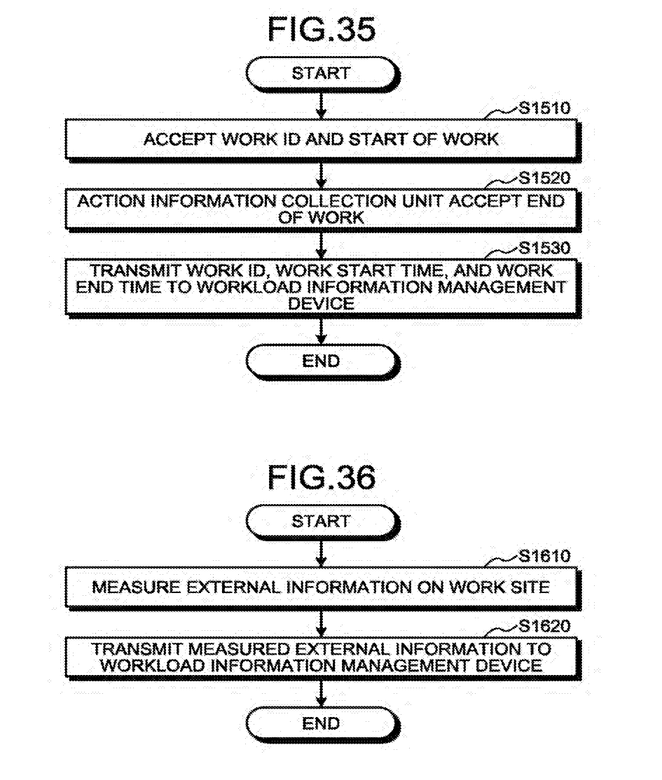

Next, an operation of the worker information collection device 3b will be described with reference to the flowchart of FIG. 35. FIG. 35 is a flowchart illustrating the operation of collecting the worker position information and transmitting the worker position information to the workload information management device 1b performed by the worker information collection device 3b according to the third embodiment.

In Step S1510, the action information collection unit 62 accepts a work ID and a work start instruction from the worker via the input unit 33. The work start instruction includes instruction information regarding what kind of work is to be performed. Upon accepting the work ID and the work start instruction, the action information collection unit 62 stores the work ID and the work start time in a memory in the action information collection unit 62. Then, the action information collection unit 62 collects worker position information. The action information collection unit 62 transmits the collected worker position information to the workload information management device 1b via the communication unit 36. The action information collection unit 62 may store the work ID and the work start time in the storage unit 35.

Next, in Step S1520, the action information collection unit 62 accepts a work end instruction from the worker via the input unit 33. Upon accepting the work end instruction, the action information collection unit 62 stores work end time in the memory in the action information collection unit 62. Then, the action information collection unit 62 ends the collection of the worker position information. The action information collection unit 62 may store the work ID and the work end time in the storage unit 35.

Next, in Step S1530, the action information collection unit 62 transmits the work ID, the work start, time, and the work end time to the workload information management device 1b via the communication unit 36.

In the procedure illustrated in FIG. 35, the action information is transmitted to the workload information management device 1b at the timing when one work operation is ended, but the transmission timing of the action information is not limited thereto. For example, multiple pieces of action information may be transmitted collectively at the timing when multiple work operations are ended. In that case, an effect is obtained where even when the worker information collection device 3b is in an environment where the worker information collection device 3b cannot communicate frequently with the workload information management device 1b, action information can be collected and provided to the workload information management device 1b.

FIG. 36 is a flowchart illustrating an operation of collecting external information and transmitting the external information to the workload information management device 1b performed by the external information measuring unit 81 of the external information collection device 8 according to the third embodiment.

In Step S1610, upon accepting a measurement start instruction via the input unit 82, the external information measuring unit 81 starts measuring external information on the work site using the sensor arranged at the work site via the communication unit 83. The sensor may be separately activated, and the external information measuring unit 81 may control the activation via the communication unit 83. The external information measuring unit 81 measures external information at a predetermined cycle.

Next, in Step S1620, the external information measuring unit 81 transmits the measured external information to the workload information management device 1b via the communication unit 83. Every time the external information measuring unit 81 measures external information on the worker at the predetermined cycle, the external information measuring unit 81 transmits the external information to the workload information management device 1b. When measuring a plurality of types of external information using a plurality of sensors, the external information measuring unit 81 transmits the plurality of types of external information to the workload information management device 1b.

Thereafter, upon accepting a measurement end instruction, the external information measuring unit 81 ends the measurement of the external information.

In the procedure illustrated in FIG. 36, the external information measuring unit 81 immediately transmits the measured external information to the workload information management device 1b, but the transmission timing of the external information is not limited thereto. For example, the external information measuring unit 81 stores and accumulates external information in the external information measuring unit 81 or a storage unit included in the external information collection device 8 until the end of the day's work, and at the timing of the end of the day's work, the external information for one day's work may be collectively transmitted to the workload information management device 1b. In that case, an effect is obtained where even when the external information collection device 8 is in an environment where the external information collection device 8 cannot communicate frequently with the workload information management device 1b, external information can be collected and provided to the workload information management device 1b.

Next, examples of operations of the worker management apparatus according to the third embodiment will be described. The worker management apparatus includes the workload information management device 1b, the worker information collection device 3b, and the workload information viewing device 4, which perform the above processes. First, an example of an operation will be described. In the operation, the worker information collection device 3b according to the third embodiment collects worker position information and transmits the worker position information to the workload information management device 1b, and then the workload information management device 1b accumulates the worker position information.

First, immediately before the worker performs work, the following procedures are executed.

(Procedure 10-1) Activation of Worker Information Collection Device 3b

When the worker who wears the worker information collection device 3b on the body turns on the power of the worker information collection device 3b, the control unit 37 of the worker information collection device 3b accepts an activation start request, and the control unit 37 activates the worker information collection device 3b.

(Procedure 10-2) Start of Collection of Worker Position Information

When the worker information collection device 3b is activated, the action information collection unit 62 collects action information and activates the position sensor to start measuring the position information on the worker.

(Procedure 10-3) Transmission of Position Information

The action information collection unit 62 transmits, to the workload information management device 1b via the communication unit 36, the measured position information on the worker together with the worker information and the collection information as worker position information.

Next, the workload information management device 1b executes the following procedures.

(Procedure 11-1) Reception of Worker Position Information

The communication unit 17 of the workload information management device 1b receives the position information transmitted from the communication unit 36 of the worker information collection device 3b. The communication unit 17 transmits the received worker position information to the action history accumulation unit 54.

(Procedure 11-2) Accumulation of Worker Position Information

The action history accumulation unit 54 stores and accumulates the received worker position information.

Next, an example of an operation will be described. In the operation, the external information collection device 8 according to the third embodiment collects external information and transmits the external information to the workload information management device 1b, and then the workload information management device 1b accumulates the external information.

First, immediately before the worker performs work, the following procedures are executed.

(Procedure 12-1) Activation of Worker Information Collection Device 3b

When the worker turns on the power of the external information collection device the control unit 84 of the external information collection device 8 accepts an activation start request, and the control unit 84 activates the external information collection device 8.

(Procedure 12-2) Start of Collection of External Information

When the external information collection device 8 is activated, the external information measuring unit 81 starts measuring external information on the work site via a sensor connected to the external information collection device 8.

(Procedure 12-3) Transmission of External Information

The external information measuring unit 81 transmits the measured external information to the workload information management device 1b via the communication unit 83.

Next, the workload information management device 1b executes the following procedures.

(Procedure 13-1) Reception of Worker Position Information

The communication unit 17 of the workload information management device 1b receives the external information transmitted from the communication unit 83 of the external information collection device 8. The communication unit 17 transmits the received external information to the external information accumulation unit 55.

(Procedure 13-2) Accumulation of External Information

The external information accumulation unit 55 stores and accumulates the received external information.

An operation in which the worker information collection device 3b collects vital sign information and transmits the vital sign information to the workload information management device 1b, and then the workload information management device 1b accumulates the vital sign information, and an operation in which the worker information collection device 3b collects action information and transmits the action information to the workload information management device 1b, then the workload information management device 1b calculates the workload value, and when the workload is prominently high compared with workload values of a plurality of people, a warning is displayed on the workload information viewing device 4 are similar to those in the first embodiment.

However, the operation in which the worker information collection device 3b in the third embodiment collects worker position information and transmits the worker position information to the workload information management device 1b, and then the workload information management device 1b accumulates the worker position information, and the operation in which the external information collection device 8 according to the third embodiment collects external information and transmits the external information to the workload information management device 1b, and then the workload information management device 1b accumulates the external information are assumed to be performed in parallel with the operations described above.

In addition to the effect of the worker management apparatus according to the first embodiment, the worker management apparatus according to the third embodiment described above can divide an action on the basis of the position of the worker and calculate the workload value for the divided action. As a result, as exemplified by a workload when the worker is staying in a specific area or a workload when the worker moves a certain distance, it is possible to compare workloads for each position situation among a plurality of people.

The worker management apparatus according to the third embodiment described above can divide an action on the basis of environmental conditions of a site where the worker performs work and calculate the workload value for the divided action. As a result, as exemplified by a workload under a high temperature exceeding a certain temperature, it is possible to compare workloads for each environmental condition of the site among a plurality of people.

Therefore, according to the worker management apparatus in the third embodiment, scenes can be extracted on the basis of position information on workers during work or information on an external environment such as a temperature of a work site, and accordingly, it is possible to know workloads on workers in consideration of the difference in working environments such as high temperature and high humidity or high-place work, even when the workers perform the same type of work.

The configurations described in the embodiments above indicate examples of the content of the present invention and can be combined with other known technologies, and part thereof can be omitted or modified without departing from the gist of the present invention.

### REFERENCE SIGNS LIST

1, 1a, 1b workload information management device; 2 wide area network; 3, 3a, 3b worker information collection device; 4, 4a workload information viewing device; 8 external information collection device; 11 action history accumulation unit; 12, 53 action segment extraction unit; 13 vital sign information accumulation unit; 14 per-scene load calculation unit; 15 per-scene load accumulation unit; 16 multiple people load comparison unit; 17, 36, 45, 83 communication unit; 18, 37, 46, 84 control unit; 31 vital sign information measuring unit; 32 action information collection unit; 33, 43, 82 input unit; 34, 41 display unit; 35, 44 storage unit; 42, 71 load information display control unit; 51 terminal information accumulation unit; 52 instruction content distribution unit; 54 action history accumulation unit; 55 external information accumulation unit; 61 instruction content display control unit; 62 action information collection unit; 81 external information measuring unit; 101 keyboard; 102 mouse; 103 microprocessor; 104 hard dish drive; 105 RAM; 106 ROM; 107 graphic chip; 108 frame buffer; 109 display; 201 touch panel; 202 sensor; 203 microprocessor; 204 main memory; 205 built-in memory; 206 graphic chip; 207 frame buffer; 208 display; 301 worker ID input area; 302 register button; 303 work selection button; 304 start button; 305 end button; 401 instruction selection button; 402 instruction content selection button; 403 submit button.

1. A worker management apparatus comprising:
a worker information collection device worn by a worker, the worker information collection device comprising:
a vital sign information measurer to collect time-series vital sign information on the worker during work; and
an action information collector to collect work information including work content of work performed by the worker and including a work start date and time and a work end date and time when the worker performs the work; and
a workload information management device capable of communicating with the worker information collection device, the workload information management device comprising:
a vital sign information accumulator to accumulate the time-series vital sign information;
an action history accumulator to accumulate action information in which worker information indicating a worker who performs the work and the work information are associated with each other;
an action segment extractor to divide a series of actions of the work recorded in the work information into a plurality of action scenes on a basis of a change in content of the action in a series of actions of the work performed by the worker, and to extract the action scenes;
a per-scene load calculator to, for the action scenes extracted by the action segment extractor, calculate a workload value that is an estimate of a workload on the worker in the action scene on a basis of the time-series vital sign information from start time to end time of an action of the worker in the action scene;
a per-scene load accumulator to, for the action scenes, accumulate the worker information, scene information for identifying the action scene, and the workload value for the action scene that are associated with one another; and
a multiple people load comparator to, for the action scenes, compare the workload value for one of the action scenes with the workload value of a plurality of people that is accumulated in the per-scene load accumulator and associated with same scene information as the scene information on the one of the action scenes and to determine whether a difference between the workload value for the one of the action scenes and a workload value furthest from the workload value for the one of the action scenes among the workload value of the plurality of people is greater than or equal to a predetermined reference value.

2. The worker management apparatus according to claim 1, comprising:
a display to display information on the worker having the difference greater than or equal to the predetermined reference value on a basis of a result of determination by the multiple people load comparator.

3. The worker management apparatus according to claim 2, comprising:
a workload information viewing device to accept instruction information instructing the worker having the difference greater than or equal to the predetermined reference value to reduce a load and to transmit the instruction information to the workload information management device, the workload information viewing device including the display, wherein
the workload information management device receives the instruction information and transmits the instruction information to the worker information collection device of the worker having the difference greater than or equal to the predetermined reference value, and
the worker information collection device receives the instruction information and displays the instruction information on a display.

4. The worker management apparatus according to claim 1, wherein
the action information collector further collects position information identifying a position of a worker,
the action history accumulator further accumulates the position information, and
the action segment extractor divides, on a basis of the action information, a series of actions of the work into the plurality of action scenes in accordance with a change in content of the action in a series of actions of the work performed by the worker and the position information accumulated in the action history accumulator, and extracts the action scenes.

5. The worker management apparatus according to claim 1, comprising:
an external information collection device to measure external information indicating an environmental condition of a site where the worker performs work, wherein
the workload information management device comprises an external information accumulator to accumulate the external information, and
the action segment extractor divides, on a basis of the action information, a series of actions of the work into the plurality of action scenes in accordance with a change in content of the action in a series of actions of the work performed by the worker and the external information accumulated in the external information accumulator and extracts the action scenes.

6. The worker management apparatus according to claim 2, wherein
the action information collector further collects position information identifying a position of a worker,
the action history accumulator further accumulates the position information, and
the action segment extractor divides, on a basis of the action information, a series of actions of the work into the plurality of action scenes in accordance with a change in content of the action in a series of actions of the work performed by the worker and the position information accumulated in the action history accumulator, and extracts the action scenes.

7. The worker management apparatus according to claim 3, wherein
the action information collector further collects position information identifying a position of a worker,
the action history accumulator further accumulates the position information, and
the action segment extractor divides on a basis of the action information, a series of actions of the work into the plurality of action scenes in accordance with a change in content of the action in a series of actions of the work performed by the worker and the position information accumulated in the action history accumulator, and extracts the action scenes.

8. The worker management apparatus according to claim 2, comprising:
an external information collection device to measure external information indicating an environmental condition of a site where the worker performs work, wherein
the workload information management device comprises an external information accumulator to accumulate the external information, and
the action segment extractor divides, on a basis of the action information, a series of actions of the work into the plurality of action scenes in accordance with a change in content of the action in a series of actions of the work performed by the worker and the external information accumulated in the external information accumulator, and extracts the action scenes.

9. The worker management apparatus according to claim 3, comprising:
an external information collection device to measure external information indicating an environmental condition of a site where the worker performs work, wherein
the workload information management device comprises an external information accumulator to accumulate the external information, and
the action segment extractor divides, on a basis of the action information, a series of actions of the work into the plurality of action scenes in accordance with a change in content of the action in a series of actions of the work performed by the worker and the external information accumulated in the external information accumulator, and extracts the action scenes.
